(19) United States
(12) Patent Application Publication
(10) Pub. No.: US 2025/0332010 A1
(43) Pub. Date: Oct. 30, 2025

(54) BLOOD VESSEL RESHAPING USING COMPRESSIBLE IMPLANTS

(71) Applicant: EDWARDS LIFESCIENCES CORPORATION, Irvine, CA (US)

(72) Inventors: Eran Grosu, Kfar-Saba (IL); Tal Reich, Moledet (IL); Yuval Kasher, Kfar Shmuel (IL); Roei Mayo, Irvine, CA (US); Gideon Meyer-Brodnitz, Ramot Menashe (IL); Gil Senesh, Adi (IL); Nikolai Gurovich, Hadera (IL); Leonardo Paim Nicolau Da Costa, Tampa, FL (US); Jenna Christine Iaizzo, San Clemente, CA (US); Anatoly Dvorsky, Haifa (IL)

(73) Assignee: EDWARDS LIFESCIENCES CORPORATION, Irvine, CA (US)

(21) Appl. No.: 19/256,351

(22) Filed: Jul. 1, 2025

Related U.S. Application Data
(63) Continuation of application No. PCT/US2024/011525, filed on Jan. 13, 2024.
(60) Provisional application No. 63/481,117, filed on Jan. 23, 2023.

(51) Int. Cl. A61F 2/90

(52) U.S. Cl. CPC A61F 2/90; A61F 2230/0008; A61F 2230/0054; A61F 2230/0069; A61F 2250/0007; A61F 2250/0039

(57) ABSTRACT

An intravascular compliance-enhancing spring implant device includes a fully-cylindrical tubular frame defining a longitudinal axis and a non-circular cross-sectional shape having a major axis diameter and a minor axis diameter that is less than the major axis diameter, first and second longitudinal tissue-contact rails associated with first and second major-axis ends, respectively, of the tubular frame and adapted to press against an inner diameter of a target blood vessel, and first and second lateral connecting strut arrays associated with minor-axis sides of the tubular frame, the first and second lateral connecting strut arrays spanning between the first and second tissue-contact rails on diametrically opposite sides of a lumen of the tubular frame and adapted to be compressed along a major-axis dimension of the tubular frame in a manner as to reduce a distance between the first and second tissue-contact rails, storing spring energy in the lateral connecting strut arrays.

### RELATED APPLICATIONS

This application is a continuation of International Patent Application No. PCT/US24/11525, filed Jan. 13, 2024, which claims the benefit of U.S. Provisional Patent Application No. 63/481,117, filed on Jan. 23, 2023, the complete disclosure of which is hereby incorporated by reference in its entirety.

### BACKGROUND

The present disclosure generally relates to the field of medical implant devices. Insufficient or reduced compliance in certain blood vessels, including arteries such as the aorta, can result in reduced perfusion, cardiac output, and other health complications. Restoring compliance and/or otherwise controlling/managing flow in such blood vessels can improve patient outcomes.

### SUMMARY

Described herein are devices, methods, and systems that facilitate the restoration of compliance characteristics to undesirably stiff blood vessels. Devices associated with the various examples of the present disclosure can include spring elements configured to be implanted within or without a target blood vessel, wherein forcible manipulation of such spring elements, such as by compression of the spring element, can store energy in the spring element that can be returned to the target blood vessel in a manner as to reshape/remodel the blood vessel and increase diastolic blood flow, thereby mimicking natural compliance of the blood vessel. In some implementations, spring implant devices of the present disclosure can be implanted within a target blood vessel segment, wherein a biased shape of the spring implant device can have a long-/major-axis dimension that forces a non-circular shape in the blood vessel, such as by pushing outward on opposite sides of the blood vessel wall to cause long-axis elongation thereof; as luminal pressure increases in the blood vessel, the blood vessel walls, at two or more contact points with the spring implant, press radially-inwardly on the spring element, thereby compressing the spring element to store spring energy in the implant and to allow the blood vessel to assume a more-circular shape. As luminal blood pressures decrease, the biased, elongated shape memory of the spring element can overcome the hoop stress/force in the blood vessel wall to once again reshape/remodel the blood vessel to the non-circular (e.g., oval) shape, wherein such cyclic reshaping/remodeling of the blood vessel can increase diastolic flow/pressure and/or decrease systolic flow/pressure.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof, Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates example cardiac and vascular anatomy of a patient having a healthy, compliant aorta.

FIGS. 2A and 2B show side and axial cross-sectional views, respectively, of the healthy aorta of FIG. 1 experiencing compliant expansion.

FIG. 3 shows an example stiff aorta.

FIGS. 4A and 4B show side and axial cross-sectional views, respectively, of the stiff aorta of FIG. 3 experiencing compromised expansion.

FIGS. 5-1 and 5-2 show a blood vessel in circular and non-circular shapes, respectively.

FIG. 6 shows a perspective view of a non-circular stent in accordance with one or more examples.

FIG. 7 shows an axial view of a non-circular stent deployed within a blood vessel in accordance with one or more examples.

FIGS. 8-1A and 8-1B show axial and perspective views of a circumferentially-open, non-circular stent in accordance with one or more examples.

FIGS. 8-2 and 8-3 show the stent of FIGS. 8-1A and 8-1B in various configurations in accordance with one or more examples.

FIGS. 9-1A and 9-1B show axial and perspective views of a circumferentially-open, non-circular stent in accordance with one or more examples.

FIG. 9-2 shows the stent of FIGS. 9-1A and 9-1B in an at least partially circularized configurations in accordance with one or more examples,

FIGS. 10-1 and 10-2 show a diametrical vessel-reshaping spring device in expanded and compressed configurations, respectively, in accordance with one or more examples.

FIG. 11 shows a vessel-reshaping spring device in accordance with one or more examples.

FIG. 12 shows a vessel-reshaping device comprising a plurality of serially-arranged spring elements in accordance with one or more examples.

FIGS. 13A-13D show perspective, side, and axial views, respectively, of a tubular vessel-reshaping spring implant device in accordance with one or more examples.

FIGS. 14A and 14B show perspective and side views, respectively, of a tubular vessel-reshaping spring implant device in accordance with one or more examples.

FIGS. 15A and 15B show perspective and side views, respectively, of a tubular vessel-reshaping spring implant device in accordance with one or more examples.

FIGS. 16A and 16B show perspective and side views, respectively, of a tubular vessel-reshaping spring implant device in accordance with one or more examples.

FIG. 17A shows a perspective view of a compressible spring stent in accordance with one or more examples.

FIGS. 17B-1 and 17B-2 show a compressible spring stent in expanded and compressed configurations, respectively, in accordance with one or more examples.

FIGS. 18A-18D show perspective, side, and axial views, respectively of a vessel-reshaping implant device configured to compress in orthogonal planes in an expanded configuration in accordance with one or more examples.

FIGS. 19A-19D show perspective, side, and axial views, respectively of the vessel-reshaping implant device of FIGS. 18A-18D in a compressed configuration in accordance with one or more examples.

FIG. 20 is a flow diagram illustrating a process for reshaping a blood vessel using a compressible implant device in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### Vascular Anatomy and Compliance

Certain examples are disclosed herein in the context of vascular implant devices, and in particular, compliance-enhancing spring implant devices implanted/implantable in the aorta. However, although certain principles disclosed herein may be particularly applicable to the anatomy of the aorta, it should be understood that compliance-enhancement implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

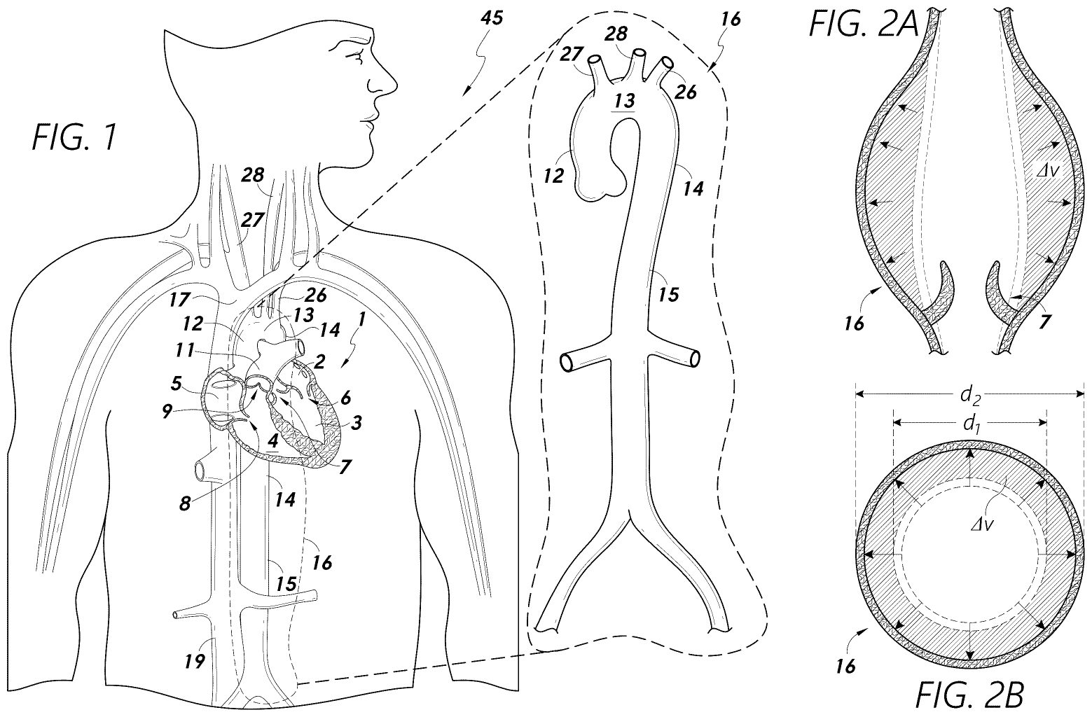

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior and superior venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery, and the right aortic sinus likewise gives rise to the right coronary artery. Together, these two arteries supply the heart.

As mentioned above, the aorta is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof. The inferior vena cava 19 generally runs parallel to the aorta 16 in the abdominal space.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

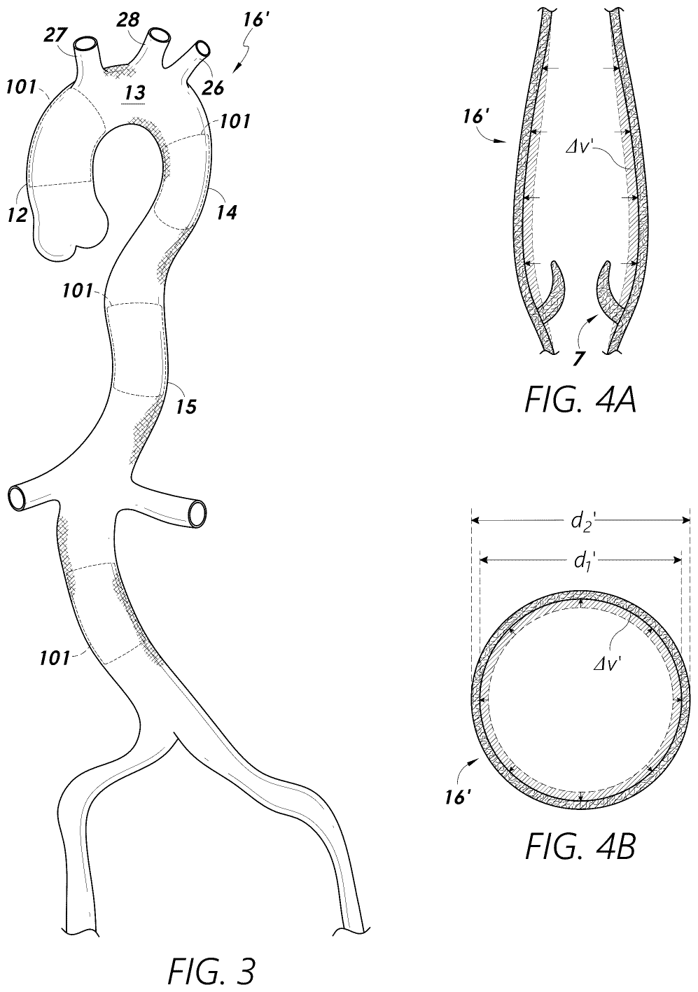

FIGS. 2A and 2B show side and axial cross-sectional views, respectively, of the healthy aorta 16 of FIG. 1 experiencing compliant expansion and contraction over a cardiac cycle. FIG. 3 shows an example stiff aorta 16′, whereas FIGS. 4A and 4B show side and axial cross-sectional views, respectively, of the stiff aorta 16′ of FIG. 3 experiencing compromised expansion and contraction over a cardiac cycle.

The systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume Av will generally occur in an artery when the pressure in the artery is increased from diastole to systole. With respect to the aorta, as blood is pumped into the aorta 16 through the aortic valve 7, the pressure in the aorta increases and the diameter of at least a portion of the aorta expands. A first portion of the blood entering the aorta 16 during systole may pass through the aorta during the systolic phase, while a second portion (e.g., approximately half of the total blood volume) may be stored in the expanded volume Av caused by compliant stretching of the blood vessel 16, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Av is the change in volume (e.g., in mL) of the blood vessel, and Δp is the pulse pressure from systole to diastole (e.g., in mmHg):

$$C = \frac{Δ ν}{Δ p} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Healthy arterial compliance may cause retraction/recoil of the blood vessel wall inward during diastole, thereby creating pressure in the blood vessel to cause blood to continue to be pushed through the artery 16 when the valve 7 is closed. For example, during systole, approximately 50% of the blood that enters the artery 16 through the valve 7 may be passed through the artery, whereas the remaining 50% may be stored in the artery, as enabled by expansion of the vessel wall. Some or all of the stored portion of blood in the artery 16 may be pushed through the artery by the contracting vessel wall during diastole. For patients experiencing arterial stiffness that causes lack of compliance, their arteries may not operate effectively in accordance with the expansion/contraction functionality shown in FIGS. 2A and 2B.

As shown in FIG. 3, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect, such as the thickening of the left ventricle muscle and/or diastolic heart failure. Stiffness in the aorta and/or other blood vessel(s) can occur due to an increase in collagen content and/or a corresponding decrease in elastin. While stiff/non-compliant blood vessels can generally suffer from a lack of elasticity in the walls thereof, as shown as causing compromised/reduced stretching and volume change Av′, such vessels can maintain some amount of flexibility/bendability, such that reshaping of the blood vessels can occur without necessarily requiring the stretching of the walls of the blood vessel.

Generally, the majority of aortic compliance is provided in the ascending aorta 12 with respect to healthy anatomy. Furthermore, calcification frequently occurs in the area of the ascending aorta 12, near the aortic arch 13 and the great vessels emanating therefrom. Such anatomical areas can experience relatively higher stresses due to the geometry, elasticity, and flow dynamics associated therewith. Therefore, implantation/deployment of compliance-enhancing, spring implant devices of the present disclosure can advantageously be in the ascending aorta 12 in some cases. While relatively less calcification tends to occur in the descending 14 and abdominal 15 aorta, implant devices of the present disclosure can advantageously be implanted/deployed in such areas as well for the purpose of increasing compliance in the aortic system. Examples of the present disclosure provide compliance-enhancing spring implant devices, which may be implanted in one or more locations in a compromised aorta and/or other vessel(s). For example, FIG. 3 shows example positions of spring implant devices 101 including features disclosed herein implanted in various areas of an aorta 16′

### Compliance-Enhancing, Vessel-Reshaping Implants

The present disclosure relates to systems, devices, and methods for adding-back and/or increasing compliance in the aorta or other arterial (or venous) blood vessel(s) to provide improved perfusion of the heart muscle and/or other organ(s) of the body. Examples of the present disclosure can include spring implant devices that, when implanted, are configured to decrease the cross-sectional area/volume of the target blood vessel segment in which the spring implant device is implanted during low-pressure conditions, such as diastole, which serves to force blood through the blood vessel segment by pushing the blood through the vessel as the vessel volume reduces in connection with spring expansion induced by cyclical drops in blood pressure.

The spring implant devices of the present disclosure can advantageously be configured to generate a differential cross-sectional area or volume of the target blood vessel(s) (e.g., aorta) between high- and low-pressure phases of the cardiac cycle to facilitate perfusion. As described above, relatively non-compliant blood vessels generally may not be able to stretch to thereby lengthen the perimeter of the blood vessel in response to increased pressure conditions. Such inability to stretch can prevent compliant expansion of the blood vessel.

As the spring implant devices of the present disclosure produce complaint blood vessel volume change by manipulating/reshaping the native blood vessel walls, compliance can be increased in the target blood vessel without requiring blood vessel grafting or resection. Therefore, compared to blood flow solutions involving blood vessel grafting/resection, examples of the present disclosure can provide a solution that avoids the risks that may be associated with cutting of the vessel and/or devices grafted in/to such vessels, which may present risk of rupture and blood leakage outside of the circulatory system. Hazards associated with extravascular arterial blood leakage, such as within the abdominal and/or chest cavity, can include the risk of serious injury or death.

As described above, desirable diastolic flow in arterial blood vessels is enabled by the decrease in cross-sectional area/volume of the blood vessels when transitioning from higher-pressure conditions (e.g., systole) to lower-pressure conditions (e.g., diastole). Where the relevant blood vessel has become stiff and non-compliant, stretching/expanding and subsequent contraction/shrinking of the blood vessel to cause the desired change in area/volume of the blood vessel may be limited due to the perimeter/wall of the blood vessel being resistant to stretching. Examples of the present disclosure provide implants that cause a change in cross-sectional area/volume of a target blood vessel without requiring stretching in the blood vessel wall. Rather, such cyclical change in blood vessel area/volume can be achieved through manipulation of the shape (e.g., cross-sectional shape) of the target blood vessel, wherein a transition between blood vessel shapes occurring in response to changing pressure conditions can reduce and increase the area/volume of the blood vessel in a cyclical manner to promote more even flow of blood through the blood vessel throughout the cardiac cycle.

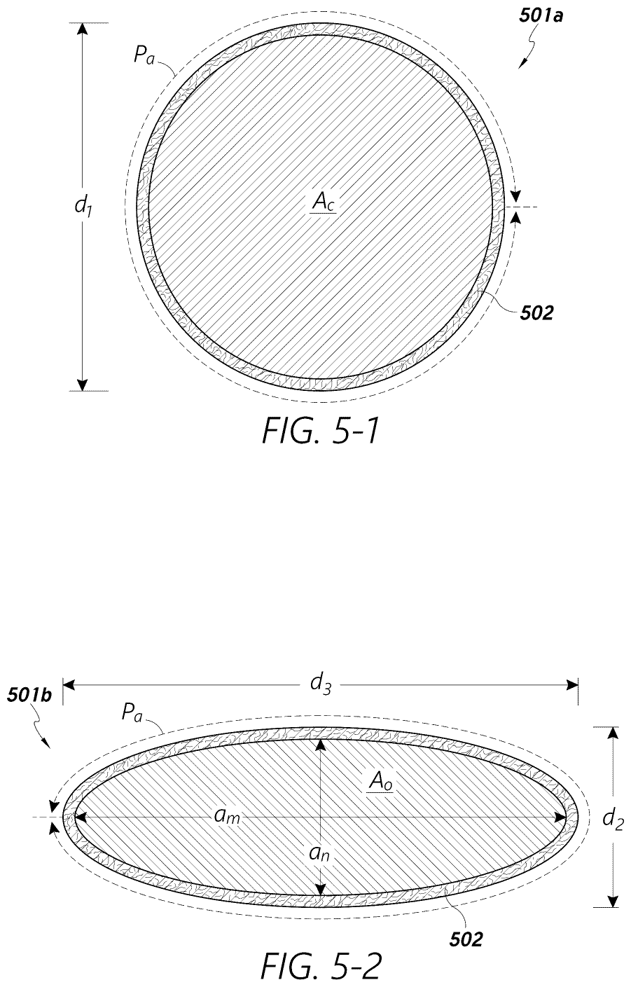

With respect to a blood vessel having a relatively fixed perimeter, wherein the blood vessel wall does not expand sufficiently due to stiffness and/or other factors of non-compliance, generally, the greatest area/volume of the blood vessel may be present/achieved when the blood vessel wall forms a circular cross-sectional shape, which may maximize the cross-sectional area and volume of the blood vessel. FIG. 5-1 shows an example blood vessel 501 (identified as blood vessel 501a in FIG. 5-1) having a blood vessel wall 502 forming a generally circular cross-sectional shape, such that the area A_{c} thereof is maximized for the given perimeter/wall-length Pₐ. In the circular configuration, the diameter d₁ is substantially constant at every angle about the axis of the vessel.

Diverging from a circular cross-sectional shape can produce a cross-sectional area/volume for a blood vessel that is less than the maximum area A_{c} shown in FIG. 5-1. For example, FIG. 5-2 shows the blood vessel 501 (identified as vessel 501b in FIG. 5-2) having a shape that resembles an oval/ellipse, which produces the cross-sectional area Aₒ that is less than the area A_{c} with the same blood vessel wall/perimeter length Pₐ. The oval shape of the vessel 501b may have a major axis aₘ having a dimension d₃ that is greater than a dimension d₂ of the minor axis aₙ thereof.

With further reference to FIGS. 5-1 and 5-2, due to the area Aₒ of the oval vessel of FIG. 5-1 being less than the area A_{c} of the circular configuration shown in FIG. 5-1, transitioning from the circular shape 501a to the non-circular shape 501b, can provide a reduction in area/volume of the blood vessel, and therefore solutions that cause transitions between circular and non-circular blood vessel shapes between cardiac phases can provide compliance characteristics without the need for elasticity in the blood vessel wall tissue. For example, where a mechanism is implemented to cause a blood vessel to transition between circular and non-circular shapes in response to changing pressure conditions, such manipulation of the blood vessel shape can introduce volumetric change in the blood vessel in response to the typical changes in pressure experienced during the cardiac cycle, thereby mimicking natural compliance to increase cardiac efficiency and reduce pulsatile load.

In view of the foregoing, examples of the present disclosure provide spring implant devices and associated processes configured to transition the shape/area of a blood vessel from circular/more-circular to non-circular/less-circular shapes, and vice versa, to enhance compliance with respect to the area of the implant reshaping. Such spring implant devices/processes may effect vessel reshaping through dynamic reshaping of the structural shape of the spring implant in a way that produces a change in shape of the blood vessel in which it is implanted to produce a change in blood vessel area/volume between the systolic and diastolic phases of the cardiac cycle. As described above, for relatively stiff blood vessels, radially-outward expansion/stretching of the blood vessel sufficient to achieve a change in volume that produces desirable compliance may not occur as pressure conditions change. Using spring implant devices in accordance with aspects of the present disclosure may be desirable to provide the necessary change in volume of the target blood vessel.

Examples of the present disclosure provide for spring-type implants that are biased to a shape that has an expanded dimension, such that, in a relaxed/non-pressurized state, the device has a greater dimension along a major axis compared to a dimension along a minor axis, wherein such implant devices are configured to transition to a compressed major-axis dimension when forcibly remodeled by with the blood vessel in which the implant is implanted that overcomes the spring bias of the spring to some degree and causes the spring element(s) to be compressed (or expanded). The ability of spring implant devices of the present disclosure to reshape the target blood vessel in the manner described above to produce the desired oval cross-section of the blood vessel can be achievable due to stiff/non-compliant blood vessels, which may be unable to stretch to a substantial degree, still retaining the ability to bend to a sufficient degree to allow for such shaping of the blood vessel. That is, the bending stiffness of a non-compliant blood vessel may be relatively lower compared to the stretching stiffness thereof. Therefore, examples of the present disclosure achieve compliance through bending energy with respect to the blood vessel wall, as opposed to stretching energy. When spring implant devices of the present disclosure are forced to a compressed/modified shape corresponding more closely to a circular shape of the target blood vessel, energy may be stored in the shape memory of the walls/struts of the implant, wherein recoil/expansion of the spring element towards its biased, long-axis configuration can return/release energy to the blood circulation.

Spring implant devices disclosed herein can be used to manage blood flow in a target blood vessel by adding some amount of compliance to the vessel. In some examples, a spring implant device in accordance with the present disclosure may provide added compliance (i.e., added change in volume over a constant change in pressure) to any blood vessel in or on which it is placed. In some implementations, spring implant devices of the present disclosure comprise stent forms. The term “stent” is used herein in accordance with its broad and ordinary meaning and may refer to any device configured to be implanted in a lumen of a blood vessel, the device having a tubular form forming a lumen through which blood can flow.

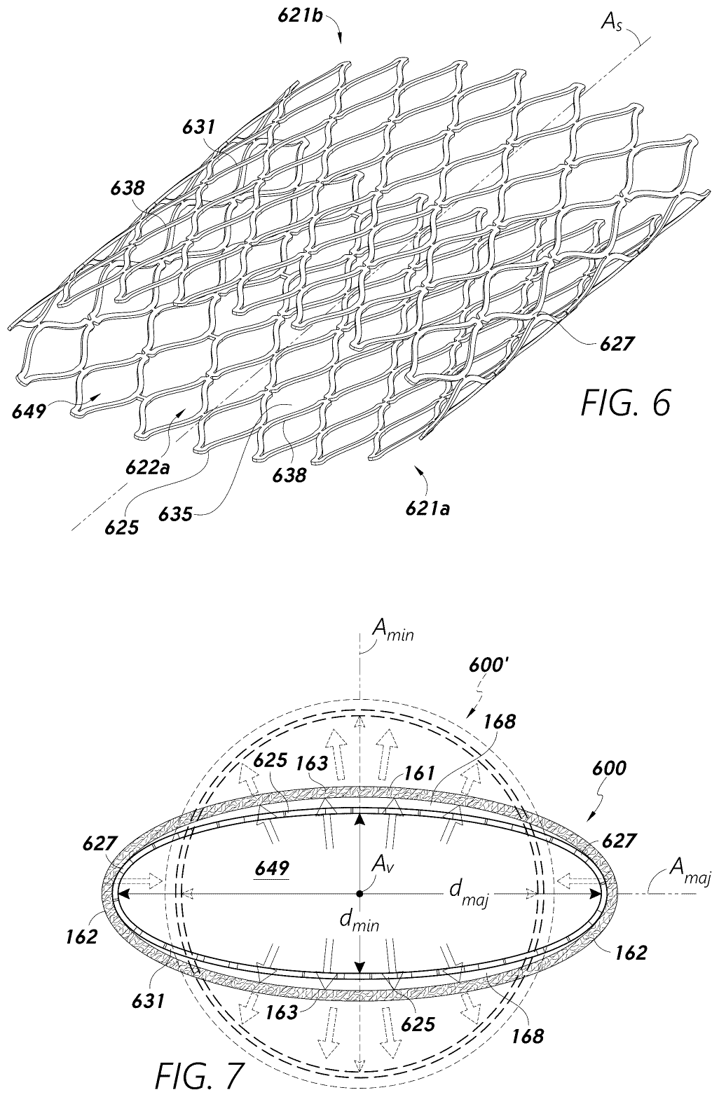

FIG. 6 shows perspective, minor-axis side, major-axis side, and axial views, respectively, of a non-circular stent 600 in accordance with one or more examples. FIG. 7 shows an axial view of the uncovered, non-circular stent 600 shown in FIGS. 6A-6D deployed within a blood vessel 61 in accordance with one or more examples. The stent 600 may be deployable within a blood vessel lumen. However, it should be understood that example stent and spring devices of the present disclosure may alternatively or additionally be deployable in a position around an outer surface of a target blood vessel. The description of the stent 600 may be understood to relate to, and/or describe aspects of, any of the spring implant devices described herein.

The stent 600, as with other spring-biased implant devices disclosed herein, may be formed of a tubular frame 631, which may form a wall around an axial channel 649, thereby defining the channel 649. As described herein, the frame wall 631 of the stent 600 can be considered a single, circumferentially-wrapped wall, or may be considered to comprise multiple contiguous walls, or wall segments. For example, the stent frame 631 may be considered to comprise sidewall segments 625 that run along relatively long sides of the stent 600 that are aligned generally with the orientation of the major axis/dimension Aₘₐⱼ of the stent, as well as end wall segments 627, which may connect the side walls 625 on major-axis ends of the stent 600. The end walls 627 may be outwardly-curved/concave with respect to an axis Aₛ of the stent 600 and may provide primary tissue-contact surfaces/portions for reshaping the target blood vessel 161. The sidewalls 625 may be generally straight over at least a portion of a length thereof, and/or may bow/deflect inward and/or outward, either in a resting, unpressurized state, or in conditions of hoop/wall stress on the frame 631. For example, the sidewalls 625 may bow outward such that the sidewalls 625 are concave from the perspective of the axis Aₛ of the stent 600 and convex from the perspective of the exterior of the stent 600, wherein the sidewalls 625 form a vertex/apex, which may be aligned with the minor axis/dimension Amin of the stent 600. The frame 631 has inlet 621a and outlet 621b openings.

Although certain oval-shaped implant devices are disclosed herein, it should be understood that the principles of the present disclosure may relate to implant devices having any non-circular shape in at least some configurations thereof (e.g., relaxed configuration). Descriptions of spring implant devices with spring elements in a relaxed configuration should be understood to relate to a configuration that the spring element naturally assumes in the absence of tension/force on the tissue-contact portions of the implant device from external forces (e.g., ambient fluid pressure, physical contact forces, etc.).

The stent 600, and certain other implant devices disclosed herein, may be considered an oval-shaped device with respect to the shape of the axial cross-section thereof, as shown in FIG. 7. The term “oval” is used herein according to its broad and ordinary meaning and may be used substantially interchangeably with the term “ellipse” and/or “oblong,” which terms are likewise used according to their broad and ordinary meanings. The term “oval” may be used to refer to any non-circular open or closed curve having major and minor axes, the major axis being greater than the minor axis. With respect to “oval”-shaped implants disclosed herein, such implants may have relatively flatter minor-axis sidewalls (compared to curved major-axis end walls), wherein the sidewalls may bow radially outward, and/or may be deflected/curved radially inward so as to produce external concavity and internal convexity in such sidewalls (e.g., forming a peanut-shaped implant). Major-axis walls of an oval implant as described herein may be considered wall portions of an implant that are intersected by a major axis of the implant that runs through an axial center of the implant. Minor-axis walls of such oval implants may be considered wall portions that are intersected by a minor axis of the implant that runs through the axial center of the implant. The description below of the various examples of implants having non-circular cross-sectional portions/sections provide further context for interpreting the terms “oval,” “peanut,” and “non-circular” in the context of oval implants and implants having oval portions/segments. Example implants of the present disclosure may be considered to have an oval shape whether or not the shape thereof is definable by an algebraic curve. Example implants of the present disclosure may be considered oval implants when the wall(s) of the implant in an axial-cross-sectional perspective form(s) a closed or open curve in a plane that is non-circular; one or more segments/areas thereof may resemble the outline of a portion of an egg. Oval implants of the present disclosure may include either one or two axes of symmetry of an ellipse, such as the illustrated major Aₘₐⱼ and minor Amin axes. The axial cross-section of some examples of oval implants of the present disclosure may resemble the union of two semicircles on opposite sides of a rectangle, providing a shape evoking the likeness of a speed skating rink or an athletics track. In some contexts, the oval implant 600 may be referred to as a “stadium”-shaped implant, or an elongated oval.

The frame 631 and/or wall(s) thereof may comprise an open-cell structure adapted to be expanded to secure the stent 600 to a blood vessel internal (or external) wall, such as through a pressure-fit deployment, one or more tissue anchors/barbs, and/or endothelialization of the frame 631 to the vessel tissue over time.

The stent 600 may be elastically deformable between a first, non-circular configuration and a second, more-circular configuration (see dashed-line representation 600′ in FIG. 7), with the stent 600 biased toward the first configuration. In some examples, the stent frame 631 may comprise a shape-memory material, such as Nitinol. Although shown as an oval-shaped stent, the stent 600 may be any non-circular shape in a resting state thereof, such as a triangle, peanut, figure-8, and/or kidney shape.

The stent 600 may be configured to be percutaneously delivered to a blood vessel in a compressed delivery configuration. Once within the blood vessel lumen at the target deployment site, the stent 600 and/or frame 631 thereof may be configured to be radially expanded into direct surface contact with the blood vessel wall (e.g., the inner wall of an aorta segment). In some examples, the stent 600 may be configured to be expanded such that the perimeter of the stent 600 approximates and/or exceeds a perimeter of the blood vessel portion where the stent 600 is implanted. The stent wall and/or a portion of the stent wall may be configured to be endothelialized to the blood vessel wall.

In the oval configuration shown in FIGS. 6A-6D, the stent 600 may have a cross-sectional area having a major/long axis diameter dₘₐⱼ that is substantially larger than the minor/short axis diameter dₘᵢₙ. For example, the major-axis diameter/dimension dₘₐⱼ may advantageously be at least twice as long as the minor-axis diameter/dimension dₘᵢₙ, or even 3, 4, 5, 6, or 7 times greater. The stent frame wall(s) 631 may be at least partially composed of struts 638 and/or stent openings/cells 635 between the struts 638.

The stent 600 may be biased toward the illustrated oval and/or other non-circular relaxed/diastolic configuration (shown in solid-line in FIG. 7), and may, when subjected to mechanical forces associated with high luminal pressure, be configured to responsively transform to a more circular systolic configuration (shown in dashed-line) such that the minor axis dₘᵢₙ approaches, and may equal, the major axis dₘₐⱼ. The cells 635 of the frame 631, formed by the arrangement of the struts 638, provide openings in the frame 631 that allow blood in the blood vessel in which the stent 600 is deployed to transfer pressure through the frame 631, to thereby load the inner diameter/surface of the blood vessel with a force resulting from increases in the luminal blood pressure as the heart beats.

Luminal pressure forces against the blood vessel wall increase the hoop stress on the blood vessel, which may force the blood vessel, and with it the stent 600, to assume a more-circular shape. The resulting hoop stress, also referred to as “tangential stress” or “circumferential stress,” from luminal pressure increase exerts radially-outward force along the blood vessel's inner circumference, such stresses/forces being tensile in nature, which can tend to cause the blood vessel to increase in diameter. However, where the elasticity of the blood vessel wall is compromised, as with the blood vessel 161, the expansion of the blood vessel diameter is limited, and therefore, the pressure increase reshapes the blood vessel without substantially increasing the circumference thereof. The blood pressure force on the blood vessel wall and resulting inward deflection of the blood vessel walls (due to outward deflection of the vessel wall portions in the area of the minor axis Amin) at the major-axis-ends 627 of the stent 600 may cause inward deflection of the ends 627 of the stent 600 to form a desired geometric change to a more-circular shape of the blood vessel and stent 600.

The transition of the stent from oval to the more-circular stent shape 600′ (shown in dashed-line in FIG. 7) causes energy to be stored in the stent frame 631 (e.g., in the elasticity and/or shape memory thereof), such that energy is returned to the blood vessel walls 62, and therefore to the blood circulation within the blood vessel segment, when the frame transitions back to the oval shape as pressure decreases. The shape memory forces of the stent 600 are advantageously sufficient to overcome the pressure forces within the blood vessel 161 to return the stent 600 to the oval configuration 600 in the presence of diastolic pressure conditions, thereby reshaping the blood vessel 161 to a non-circular (e.g., generally-oval) cross-sectional shape. With respect to implantation within the aorta or other arterial blood vessel, the systolic phase of the cardiac cycle, during which pressure levels in the aorta/arteries are relatively higher, causes the transition of the blood vessel 161 and stent 600 to the more-circular shape (shown in dashed-line in FIG. 7), whereas the diastolic phase, which is associated with relatively lower arterial blood pressure levels, causes the elongation of the stent in the major axis Aₘₐⱼ dimension to the lower-energy oval configuration, thereby forcing the blood vessel 61 to likewise assume a more oval shape due to the blood vessel having a perimeter/circumference that is sufficiently close to the perimeter/circumference length of the stent 600 (e.g., within 20% of the length of the perimeter/circumference of the stent 600).

The natural cross-sectional shape of the aorta (and other blood vessels) may generally be circular; as explained above, for a given blood vessel wall circumference/perimeter length, the circular configuration of the blood vessel may provide the maximum area/volume within the respective blood vessel segment. Therefore, any deviation from such circular/cylindrical form of the blood vessel wall may decrease the area/volume within the respective blood vessel segment. With the oval stent 600 pushing outward to the oval configuration, the wall portions 163 may be pulled/drawn at least partially towards an axial center Aᵥ of the blood vessel 161 and/or towards each other in a manner as to cause the blood vessel 161 to form a non-circular/-cylindrical shape, such as the oval shape shown in FIG. 7. Depending on the relative size of the stent 600 to the vessel 161, the blood vessel 161 may not necessarily conform exactly to the circumference and/or shape of the oval stent 600, and some minor gap(s) 68 may be present and/or form between the frame 631 and the blood vessel wall 163 as the luminal pressure increases and pushes the vessel side walls 163 away from the frame sidewall 625.

The luminal pressure exerts radial outward force against the vessel internal wall, wherein such forces indirectly act against the major axis dₘₐⱼ of the stent 600 to force outward deflection of the sidewalls 625 towards the circular shape 600′. For example, as the blood pressure increases in the vessel 161, the hoop stress on the blood vessel walls may force the side wall portions 163 of the blood vessel 161 to deflected radially outward towards a more uniform circular shape of the blood vessel 161, wherein the stiffness of the blood vessel wall causes the outward deflection of the sidewalls 163 to pull radially inward on the end walls 162 of the blood vessel, thereby applying radially-inward pressure on the end walls 627 of the stent frame 631. Such pressures force inward deflection of the walls 627 of the stent 600, thereby allowing the sidewalls 163 of the blood vessel 161 to deflect outwardly towards the circle dimension. Therefore, with a bare-frame stent, transition force for transitioning the stent to a circular shape from an oval shape may necessarily be implemented from two primary contact points in the areas of the sidewalls 627 and/or vertices associated with the major-access ends of the stent frame 631. Such forces inwardly compress the major axis dₘₐⱼ of the stent 600. As the pressure in the blood vessel 61 increases (e.g., in connection with the systolic phase of the cardiac cycle), the plastically-deformable nature of the stent 600 allows for the sidewalls 625 of the frame 631 to be pushed outward to accommodate the shortening of the stent 600 in the major axis dimension Aₘₐⱼ. As with any of the examples disclosed herein, the implant 600 can be configured to deflect from the elongated/oval shape to a more-circular shape in the presence of threshold blood pressure levels greater than 80 mmHg, such as blood pressure levels greater than 90 mmHg (e.g., between 90-120 mmHg).

### Circumferentially-Open Vessel-Reshaping Stents/Clamps

As described in detail above, stent devices can be used to reshape target blood vessels in a cyclical manner to thereby alter the blood vessel volume during the cardiac cycle in a manner as to improve and/or even-out blood flow therein. In some implementations, oval or other non-circular stents/implants can be configured with struts/cells along the entire circumference of the stent, as in FIGS. 6 and 7. Other examples relate to stent implant devices that are at least partially open with respect to a circumference or perimeter of the stent, wherein such opening/gap may extend longitudinally/axially with respect to the axis orientation of the stent. Such solutions can provide improved cardiac blood flow through blood vessel reshaping while minimizing or reducing interference with blood flow within the target blood vessel. Circumferentially-open implant devices of the present disclosure can comprise C-shaped stents, which may be considered to have the form of a clamp or other similar structure. Such radially-/circumferentially-open stents/implants can be configured as intravascular and/or extravascular implants, wherein such implants may be designed to facilitate the transition of the target blood vessel section (e.g., aortic section) between non-circular (e.g., oval) and more-circular configurations, thereby restoring at least some amount of compliance to the target blood vessel while reducing blood flow interference.

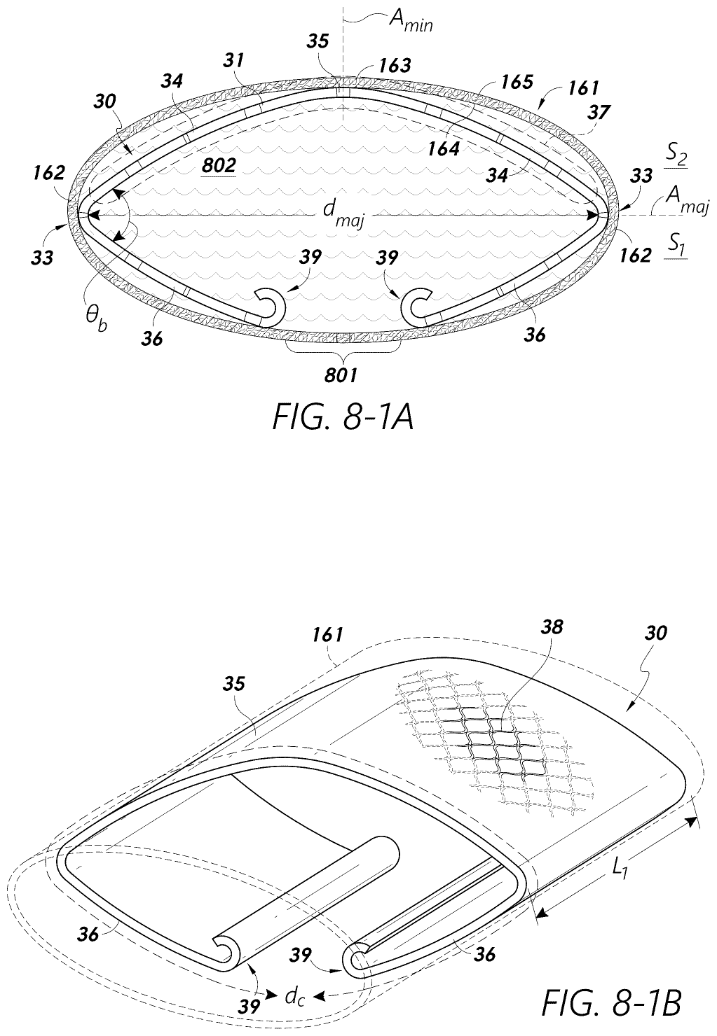

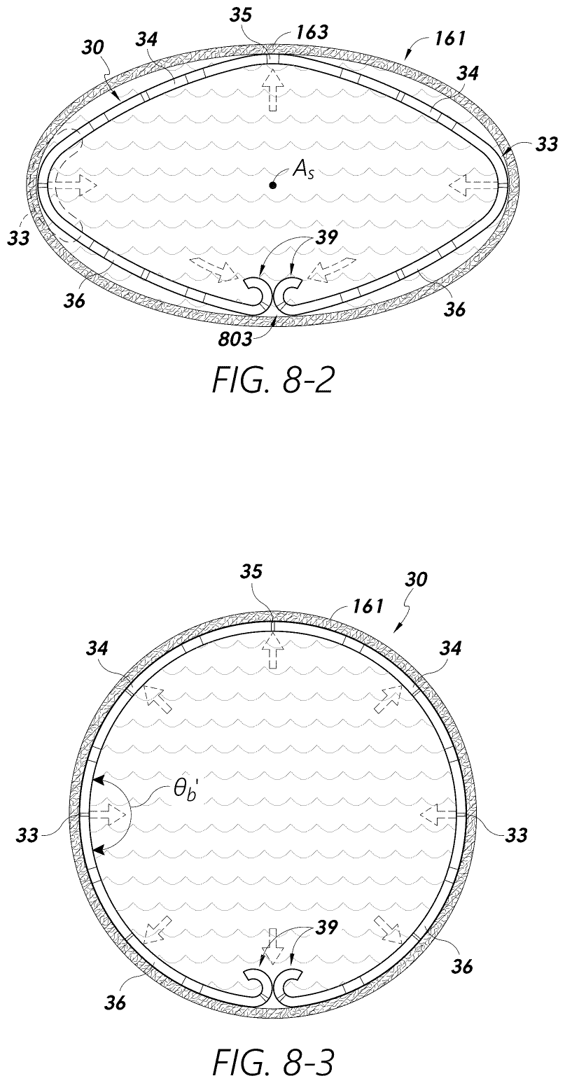

FIGS. 8-1A and 8-1B show axial and perspective views of a circumferentially-open, non-circular stent 30 in accordance with one or more examples. The stent 30 may be considered a C-shaped implant device, and may be configured to be disposed within the lumen of a target blood vessel segment 161, such as a non-compliant vascular/aortic section. The stent implant can be spring-biased, for example by being shape-set from a shape-memory material (e.g., nitinol). In FIG. 8-1A, the stent 30 is shown in a relaxed, low-pressure configuration/state within the target blood vessel 161.

The stent implant 30 may comprise a planar form, such as a planar body/sheet, that is wrapped to form an internal channel/conduit 802, wherein the stent 30 is not fully circumferentially wrapped, but rather circumferential ends/edges 39 thereof define a gap 801 in the perimeter/circumference of the stent 30. The form of the stent 30 may comprise a stent frame 31 including a plurality of struts 38 forming open cells, as described in detail herein. However, the stent 30 is shown in FIG. 8-1B primarily as a planar sheet/form for the purpose of visual clarity. It should be understood that the stent 30 may be formed of any structure, such as a stent frame, a sheet of metal, plastic, or other at least partially rigid material, or any other structure or form. The stent 30 may have any suitable or desirable length Li, such as a length between 2-10 cm.

The stent 30 may have a non-circular axial cross-sectional shape, in a relaxed configuration thereof, as shown in FIG. 8-1A. For example, the cross-sectional shape of the stent 30 may be at least partially oval/elliptical in shape, and/or may include one or more bends or contours configured to produce the desired reshaping effect of the stent 30. A particular example cross-sectional shape is shown in FIGS. 8-1A and 8-1B, which comprises an open diamond shape, wherein the non-circular shape of the stent 30 includes major-axis bends/apices 33 from which free arms, tabs, or flaps 36 extend on one diametrical side S, at an angle θ; relative to the back portion 37 of the stent 30. The open gap 801 may be positioned opposite an apex 35 of the back portion/wall 37, which extends circumferentially between the elbow bends 33. While described herein as a diamond shape, it should be understood that the shape of the stent 30 may be, or may be considered to be, any oval- or diamond-type shape, such as the shape of a leaf, fish, flame, pointed oval, ellipse, marquise, navette, mace head, vesica piscis, or the like.

FIG. 8-1B shows the circumferential dimension de of the stent 30. With respect to such dimension, the circumferential ends/edges 39 of the circumferential arms 36 are positioned approximate to one another without touching in a relaxed configuration of the stent 30, thereby maintaining the circumferential gap 801. The circumferential bends/apices 33 may be coupled to and/or integrated with the back panel/wall 37, which may have a minor-axis apex 35, wherein wall portions 34 extend circumferentially between the minor-axis apex 35 and the major-axis elbow bends/apices 33. The wall portions 36, 34 may be curved and/or at least partially straight. The various bends 33, 35 may be generally parallel with one another and with the axis of the stent 30. The minor-axis bend 35 may be positioned as a central bend of the back portion 37, as shown.

When implanted, the circumferential elbow/major-axis bends 33 may be configured to press against an inner diameter/surface 164 of wall portions 162 of the target blood vessel 161, whereas the back/minor-axis bend 35 may contact a wall portion 163 of the blood vessel 161 that is positioned circumferentially between the side wall portions 162. Although the back bend/apex 35 is shown as contacting the inner diameter of the blood vessel 161, in some implementations a radial gap is present between the back wall 37 of the stent 30 and the blood vessel wall 163 at least during a relaxed state of the stent 30.

The major-axis bends 33 can be positioned at opposite circumferential portions of the frame/body 30, wherein the bends 33 define the major-/long-axis dimension/diameter dₘₐⱼ (e.g., longest diameter) of the stent 30. The bends 33 further may provide the primary tissue-contact surfaces/portions of the stent 30.

The stent 30 can be spring-biased, for example by being shape-set to the relaxed/biased shape shown in FIG. 8-1A. When implanted, the bends 33 of the stent 30 are configured to press against opposite sides 162 of the target blood vessel wall in a manner as to cause the blood vessel 161 (e.g., aorta) to assume an oval shape as shown in FIG. 8-1A, at least during low-pressure conditions (e.g., diastole). The biasing force of the C-shaped implant 30 can be selected/set to produce a shape and/or configuration that presses at both sides/bends 33 against the blood vessel walls 162, thereby forcing the blood vessel to assume the oval-shaped configuration shown in FIG. 8-1A by contact-pressing the blood vessel at both major-axis ends 33 of the stent 30. The shape of the stent 30 may further be configured such that during elevated pressure conditions, such as during systole, luminal pressures acting against the inner diameter 164 of the blood vessel wall, and/or against the inner diameter of the stent 30 in implementations in which a fluid-tight covering is disposed without or within the frame/structure of the stent 30, overcome the spring force of the stent 30 and allows the blood vessel section 161 to assume a more-circular shape, thereby compressing the C-shaped stent/implant 30. As the luminal pressure is lowered, such as during diastole, the C-shaped implant 30 can revert to its relatively flatter/narrower biased state, thereby reverting the blood vessel segment back to the non-circular/oval shape and pushing blood flow through the target blood vessel segment as a consequence of such reshaping. In implementations including a covering, as described in detail herein, the covering may span the entire circumference of the device 30, including the gap 801, or may end at or near the ends 39, thereby maintaining the gap 801 without any covering spanning such gap.

FIGS. 8-2 and 8-3 show the stent of FIGS. 8-1A and 8-1B in various configurations in accordance with one or more examples. In particular, FIG. 8-2 shows an intermediate state/configuration of the stent 30, which may be assumed as a transitional state between the biased shape shown in FIG. 8-1A and the more-circular high-pressure state/configuration shown in FIG. 8-3. For example, as luminal pressures increase from the diastolic pressure towards the higher systolic pressure, luminal pressure forces on the inner diameter of the blood vessel 161 and/or on the structure of the stent 30 may initially cause the bends/apices 33 of the stent 30 to be deflected radially inward towards an axis Aₛ of the stent 30, which may push the free/open arms 36 towards one another until the edges/ends 39 thereof come into apposition/contact 803 with one another. In some examples, the ends 39 of the arm segments 36 may be curved, thereby presenting convex rounded surfaces, which may reduce the risk of the edges/ends 39 causing abrasion or other damage to the blood vessel wall and/or becoming caught/impeded by the blood vessel wall. That is, the curved shape of the end portions 39, which may be produced by radially-inwardly deflecting the free edges of the end portions 39 and curling the edges back towards the stent frame/structure, may provide atraumatic surfaces/ends for the arms 36. Furthermore, the curved surface of the hands 39 may produce convex contact surfaces of the respective ends 39 that facilitate force contact between the opposing ends/edges 39 when the ends are in physical contact with one another.

When implanting the intravascular implant 30, the gap 801 may advantageously be oriented/aligned with one or more side branches branching off from the target blood vessel segment 161, thereby permitting blood flow from the target blood vessel into the side branch(s), at least during certain stages/phases of the cardiac cycle (e.g., during diastole). That is, the gap 801 may be positioned to provide access to an ostium or ostia of one or more arterial branch vessels. Although the stent 30 is shown as having a rounded diamond shape, it should be understood that the stent 30 may be implemented with a more-oval shape, wherein the bends/apices thereof are more rounded than those illustrated in FIGS. 8-1-8-3.

Once the ends/edges 39 have come into contact with one another due to inward deflection of the bends/apices 33, further inward deflection/force against the bends/apices 33 may cause radially-outward bowing/deflection of the relatively flat wall portions 34, 36 of the stent 30, thereby transitioning the stent from the rounded diamond shape shown in FIG. 8-2 to a more rounded/circular form. FIG. 8-3 shows the stent 30 in the more-circularized high-pressure state/configuration. In transitioning between the intermediate configuration of FIG. 8-2 to the more-circularize configuration of FIG. 8-3, forces causing inner deflection of the bends/apices 33 may produce the outward bowing/deflection of the walls 34, 36, which may further produce outward deflection of the back bend/apex 35 and/or the contacting edges/ends 39. As the walls 34, 36 become bowed radially outward and more curved, the bends/apices 33 and/or 35 may likewise become more rounded. That is, the angle between Ob the walls 34, 36, and the radius of curvature of the bends/apices 33, 35, may increase as the walls 34, 36 bow outward, thereby producing an axial cross-sectional shape that approximates that of a circle, thereby producing a greater volume/area of the blood vessel in the segment that is reshaped by the stent 30. The hoop stress in the blood vessel 161, in high pressure conditions, can forcibly change the internal bend angles θ_{b} to the increased angle θ_{b′}. The stent 30 allows the target blood vessel segment 161 to transition between more-ovalized and more-circular configurations, thereby restoring some amount of compliance to the blood vessel segment 161, which may otherwise be relatively non-compliant or less-compliant.

When the stent 30 is transitioned to the more-circular shape shown in FIG. 8-3, the stent 30 may be in full circumferential contact with the inner vessel wall, with little or no radial gaps between the stent and the blood vessel wall. Although the term ‘C-shaped’ is used herein to describe the shape of the stent 30 and similar examples, it should be understood that such devices are not necessarily completely curved, but rather the term ‘C-shaped’ may refer to any shape that does not enclose a complete perimeter, but rather comprises two end/edge portions that are spaced out from one another, at least in a biased configuration thereof, such as an open diamond-shaped, rectangle-shaped and/or other-shaped implementations.

The contact points between the stent 30 and the inner diameter of the blood vessel 161, when the stent 30 is in the biased configuration shown in FIG. 8-1A, may include the major-axis bends/apices 33 and/or the back wall bend/apex 35. Such tissue-contact points can advantageously provide effective arterial contact pressures, while producing suitable and/or reduced interference with blood flow due to the limited perimeter spanned by the stent frame, such as compared to a full circumferential stent. By including the bends associated with the various apices 33, 35 of the stent form, the stent 30 may provide the desired spring forces. That is, compared to more rounded/curved configurations/implementations, the circumferential bends/apices of the stent 30 may have a relatively greater spring forces, wherein such spring forces may be provided primarily in the relatively sharp bends of the 33 and/or 35.

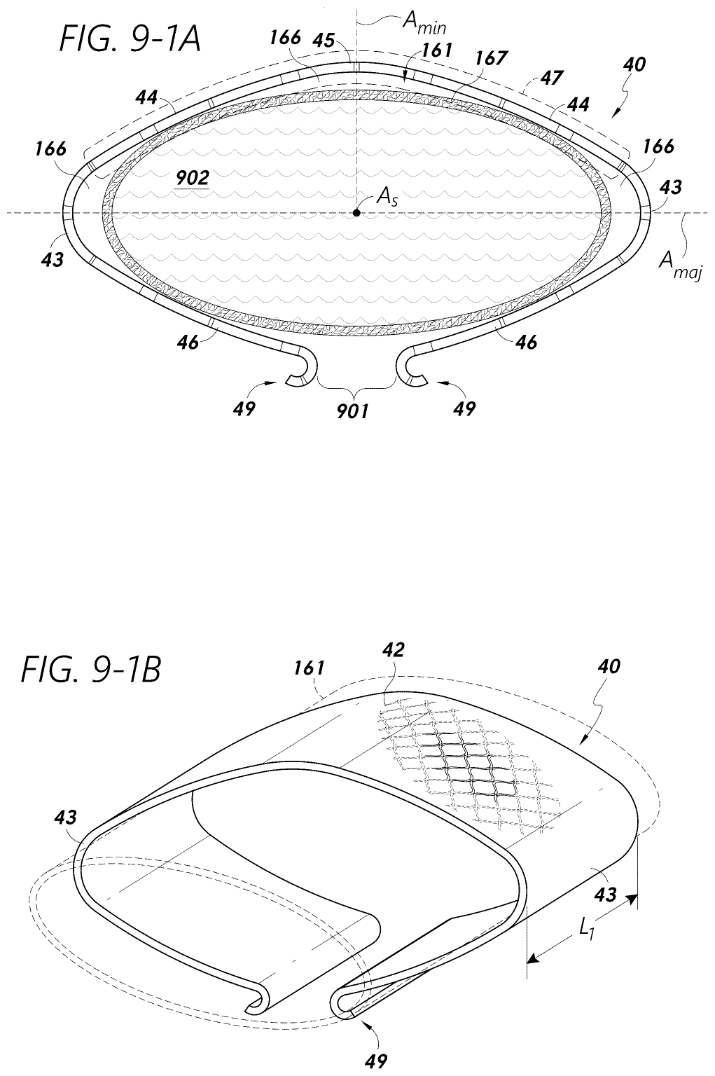

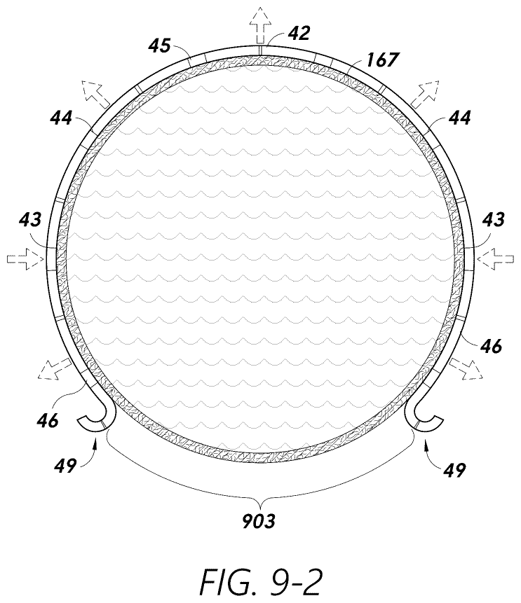

While the examples of FIGS. 8-1-8-3 are shown and described as being implantable within an interior lumen of a target blood vessel segment, it should be understood that C-shaped implants of the present disclosure may be implanted in an extravascular contact with a target blood vessel. FIGS. 9-1A and 9-1B show axial and perspective views of a circumferentially-open, non-circular stent 40 implanted around an outer wall 167 in accordance with one or more examples.

The extravascular stent implant 40 can be designed to function and a similar manner to that described above with respect to the intravascular implant 30 of FIGS. 8-1-8-3. However, the extravascular implant 40 may provide certain advantages with respect to avoiding interference with blood flow within the lumen 902 of the target blood vessel 161 due to the implant 40 being disposed around the blood vessel 161 rather than within.

The implant device 40 may be understood to include any of the features of the stent 30 described above in connection with FIGS. 8-1-8-2. For example, the stent 40 may include a back portion 47 spanning between elbow bends 43, from which free arms 46 emanate that terminate with end portions 49 including rounded/convex atraumatic contact surfaces. The stent 40 can be shape-set in a biased/relaxed configuration in which the free edges 49 are spaced to define an open circumferential gap 901. The back wall 47 can include a central bend/apex 45 that is positioned circumferentially opposite the gap 901, wherein relatively flat wall portions 44 extend between the back bend 45 and the side elbow bends 43.

In some implementations, when the stent 40 is implanted around a target blood vessel 161, gaps 166 may be present between the apices 43 and/or 45 of the stent 40 and the outer wall 167 of the blood vessel 161 when in the low-pressure configuration shown in FIG. 9-1A. That is, the reshaping of the blood vessel 161 in such configuration may not produce stretching of the blood vessel that fully fills the major-axis Aₘₐⱼ contours of the bends/apices 43 of the stent 40 and/or the minor-axis Amin contour of the back apex 45 in the ovalized shape of the blood vessel. The contact between the stent 40 and the outer wall 167 of the blood vessel 161 may generally be in the areas of the wall segments 44 between the bends 43 and the bend 45 and/or the arm wall segments 46 extending between the bends 43 and the open edges/ends 49.

In the extravascular example of FIGS. 9-1 and 9-2, the contact points between the stent 40 and the blood vessel wall 167 can advantageously be relatively large areas, which may produce less stress on the arterial wall. Furthermore, the radially-inward pressing of the outer blood vessel wall 167 may produce less stress on the blood vessel wall compared to radially-outward stretching associated with intravascular implementations of C-shaped stent implants as described herein. Furthermore, by implementing a circumferentially-open stent as an extravascular implant device, the gap 901 of the device may facilitate opening of the stent 40 to place the stent around the target blood vessel 161 in a manner that may not be feasible with respect to closed stent implants.

Whereas the end/edge portions 39 of the implant 30 described above in connection with FIGS. 8-1-8-3 were shown and described as including edges that were deflected radially inwardly to produce the desired atraumatic contact surfaces for the ends 39 of the open stent arms 36, with respect to the extravascular implant 40 of FIGS. 9-1 and 9-2, the ends 49 may advantageously be curled/deflected radially outwardly from the axis Aₛ of the stent 40. That is, because the blood vessel is disposed radially within the stent form 40, radial outward deflection of the edges of the end portions 49 may distance such edges from the blood vessel 161, thereby producing the desired reduced risk of abrasion and/or catching on the exterior walls 167 of the blood vessel 161.

FIG. 9-2 shows the stent of FIGS. 9-1A and 9-1B in an at least partially circularized configuration in accordance with one or more examples, wherein such circularization may be in response to elevation and luminal pressure within the blood vessel 161. For example, as shown in FIG. 9-2, as the luminal pressures within the blood vessel 161 increase, such pressure forces may overcome the spring forces of the extravascular stent 40, thereby allowing the blood vessel to form a more-circular shape by outwardly deflecting the wall portions 44, 46, causing the bends/apices 43 and 42 to become more rounded to conform to the more-circular shape of the blood vessel 161. In some implementations, the circularization of the blood vessel 161 may produce the above-described circularization/rounding of the stent 40, which may cause the ends 49 of the arms 46 to be pulled farther apart from one another, thereby producing an increased circumferential gap 903 between the ends 49.

Although FIG. 9-1A shows the blood vessel assuming a generally-oval shape in response to inward compression by the walls 44, 46 of the stent 40, it should be understood that in some implementations, the blood vessel 161 can conform more closely to the shape of the stent 40. For example, the blood vessel 161 may form a rounded diamond shape resembling that of the stent 40 in some cases.

### Longitudinally-Compressible Intravascular Spring Implants

Various blood-vessel-reshaping implant devices are disclosed herein, wherein some such devices are configured to reshape a target blood vessel through direct contact between the implant device and one or more portions of an inner or outer blood vessel wall. It can be desirable to design vessel-reshaping implant devices in a manner such that they are not only effective with respect to the blood vessel reshaping effects thereof, but also provide reduced or minimized contact between the implant device and the native blood vessel anatomy. For example, contact between reshaping implant devices and native blood vessel anatomy, over many heart cycles, can produce abrasion, irritation, and/or other damage to the blood vessel anatomy, depending on the type of contact, the number of contact points, the contact surface(s), and/or the like. In some implementations, examples of the present disclosure provide spring-type implants that are configured to reshape a target blood vessel through contact in only two interior blood vessel contact points or areas, such as points/areas on opposite diametrical sides of the target blood vessel. In some implementations, aspects of the present disclosure relate to intravascular implant devices that comprise longitudinally-compressible spring features/elements, wherein such spring features are configured to effect stretching/reshaping of the target blood vessel from within the blood vessel by elongating a major axis of the blood vessel, thereby shaping the blood vessel into a stretched-out, ovalized shape.

FIGS. 10-1 and 10-2 show a diametrical vessel-reshaping spring device 50 in expanded and compressed configurations, respectively, in accordance with one or more examples. The implant 50 shown in FIGS. 10-1 and 10-2 represents an example blood-vessel-reshaping device comprising one or more spring-biased elements 51, wherein the device 50 is implantable within, for example, a non-compliant blood vessel segment 161. The example implant 50 is an example of a type of implant of the present disclosure that is designed to facilitate transition of a target blood vessel segment between oval and more-circular configurations, thereby restoring and/or increasing compliance characteristics thereof, while advantageously maintaining only two points of contact between the implant and the inner wall of the target blood vessel.

The implant device 50 comprises one or more springs 51 dimensioned to extend across a blood vessel lumen in a biased shape thereof. The spring(s) 51 can be any type of compression, extension, or torsion spring(s).

Although a two-dimensional representation of the spring implant device 50 is presented in FIGS. 10-1 and 10-2, it should be understood that the implant 50 may have an axial width dimension that is parallel with an axis of the blood vessel 161. That is, with respect to the illustrated orientations of FIGS. 10-1 and 10-2, the implant 50 may extend in and/or out of the page some distance/length, such as a length of between 1-10 cm, or longer. In such implementations, the implant 50 may effectively span a longitudinal segment of the target blood vessel, thereby potentially enhancing the reshaping effect of the implant device on the blood vessel 61.

The spring implant device 50 may comprise a plurality of springs in some implementations. For example, as shown, the implant 50 may comprise a first spring 51a associated with a first longitudinal side of the spring element 54. The spring 51a may be coupled to and/or disposed/positioned adjacent to a first tissue contact end 53a, whereas the second spring 51b may be associated with a second longitudinal side/half of the implant 50 and coupled to or disposed/positioned adjacent to a second tissue contact end 53b. In implementations comprising multiple springs, the springs may be coupled at a central connector portion 52. For example, the central portion 52 may or may not have spring feature(s) associated therewith. In some implementations, a single spring spans the entire medial/spring portion 54 of the spring implant device 50.

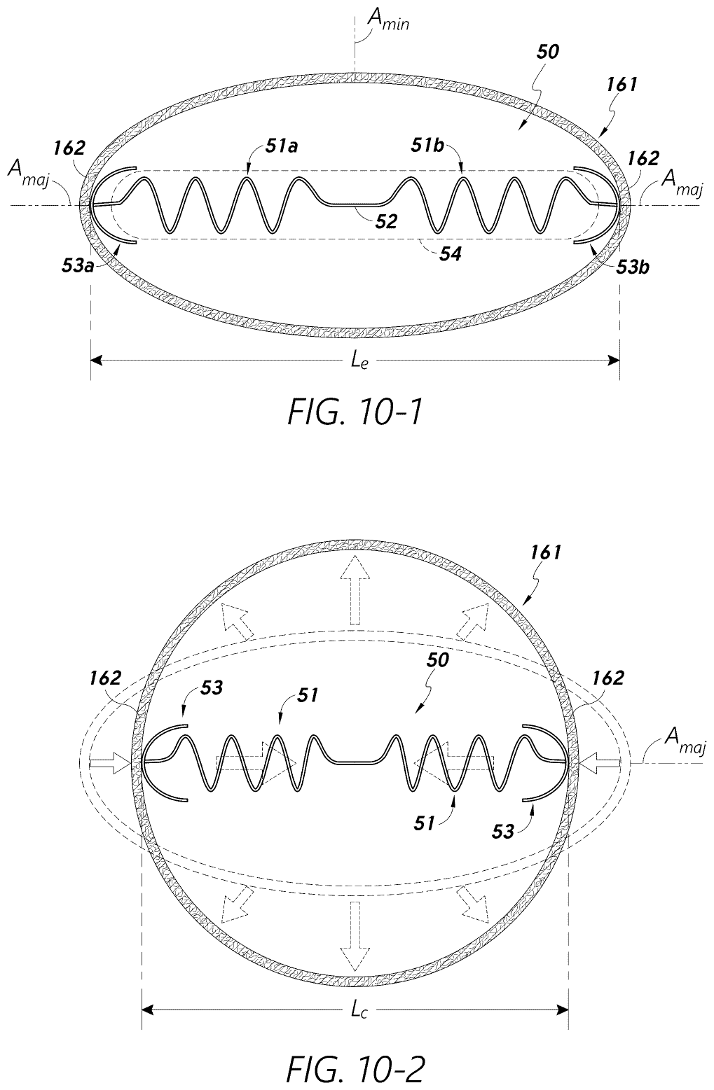

The spring implant 50 may comprise tissue-contact features 53 associated with opposite ends of the device 50. The ends 53 may have atraumatic tissue-contact surfaces facing longitudinally outward with respect to the orientation of the spring element 54, such that the tissue-contact ends 53 are opposite-facing with respect to one another. For example, the tissue-contact ends 53 may comprise curved sleds or other curved surfaces, as illustrated, wherein the curvature of the surfaces may advantageously provide reduced risk of damage to the inner wall of the blood vessel 161, while providing sufficient tissue contact surface to allow the implant 50 to stretch-out the blood vessel as in FIG. 10-1. The curvature of the ends 53 may further allow sidewalls 162 of the blood vessel to curve in an oval-shaped manner, as shown, without being unduly impeded in such shaping by the presence of the ends 53.

The spring implant 50 may have a relaxed elongated configuration, as shown in FIG. 10-1. For example, the springs 51 of the device 50 may be biased to a relatively elongated configuration, thereby producing an expanded length Le of the spring implant device, wherein luminal pressures within the blood vessel 161 sufficient to overcome the spring forces of the spring(s) 51 to some degree can produce shortening of the implant 50 to a shortened length Le, as the walls 162 on opposite sides of the blood vessel 161 exert inward radial force on the spring contacts 53, thereby compressing the springs 51, as shown in FIG. 10-2. The cyclical reshaping of the blood vessel 161 by the spring implant device 50 may involve repetitive expansion of the implant 50 to ovalize the blood vessel 161 and compression of the implant 50 as the blood vessel circularizes in high-pressure conditions, as in FIG. 10-2.

As demonstrated in FIGS. 10-1 and 10-2, the spring-biased implant device 50 is configured to impart an oval-shaped configuration of the target blood vessel segment 161 in which the implant 50 is implanted, for example during diastole or other low-pressure state/condition, and allow the blood vessel 161 to transition to a more-circular shape during systole as the luminal blood pressure increases to a degree that overcomes the spring force of the spring element(s) 54. For example, as the luminal blood pressure increases, such pressure may produce hoop stress in/on the blood vessel wall, thereby elongating the minor-axis diameter of the blood vessel 161, which in turn causes a decrease in the major-axis diameter of the blood vessel 161 due to the fixed or reduced-elasticity characteristic of the blood vessel wall perimeter.

As described above, the implant device 50 may comprise any suitable spring means/mechanism 54, such as a coil compression spring, or other element configured to store potential energy in the form of spring force when compressed and/or elongated. Furthermore, although a single plane and row of spring element(s) 54 is shown, it should be understood that spring implant devices of the present disclosure may have parallel planes and/or rows of spring element(s) in connection with any of the disclosed examples of the present disclosure.

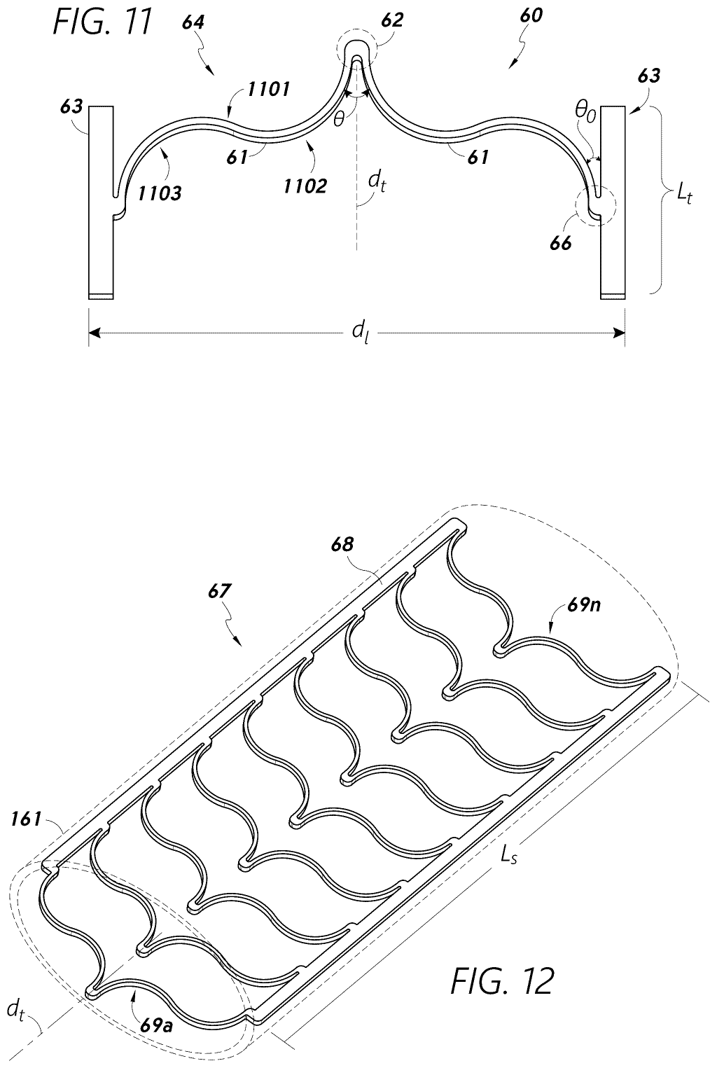

FIG. 11 shows a vessel-reshaping spring device 60 in accordance with one or more examples. The spring device 60 may be considered a V-shaped, or wishbone-shaped spring device. That is, as an alternative to other examples that comprise elongated coil spring elements having an axis parallel with a stretched major-axis of the target blood vessel, the spring element 64 of the implant 60 comprises a V-spring having a shape configured to promote bending thereof primarily at an apex 62 of the spring element 64, wherein the spring arm 61 and apex 62 are shape-set, such that bending of the apex 62, such as by compressing the spring element 64 along a lateral dimension d₁ to thereby decrease an angle θ of the apex bend 62, causes energy to be stored in the spring element 64, and particularly in the apex/bend 62.

Compared to other coil-type springs and/or other vessel-reshaping implant solutions, the V-shaped spring element 64 of the implant 60 may provide improved structural stability under compression, thereby reducing the risk of failure/breakage of the spring element over time. For example, certain coil-type spring implant devices may not provide suitable or desirable stability under compression. However, with respect to the V/wishbone shape/configuration of the implant 60, the bend/apex 62 of the spring element 64 is configured to flex in the dimension d₁ that is parallel with a resulting long/major axis of the ovalized target blood vessel segment when reshaped, wherein the apex spring 62 may be relatively robust due to material characteristics thereof, which may comprise nitinol or other metal/alloy. The spring constant of the apex spring 62 and/or other bend/portion of the spring element 64, which may be dictated at least in part by the material properties of the device and/or the shape/thickness of the arms 61, may be selected/determined such that during diastole or other low-pressure condition, the ends 63 of the device 60 are pressed against the inner blood vessel wall, thereby forcing the blood vessel to assume an oval-shaped configuration, while elevated pressure levels, acting against the aortic wall, can overcome the spring force of the spring element 64 and allow the aortic/blood-vessel segment to assume a more circular shape, thereby compressing the V-shaped spring element 64.

The spring element 64 of the implant device 60 may include a plurality of curved (e.g., S-shaped, as shown) arms/struts 61 disposed on either side of the central apex 62. The opposing tissue-contact ends 63 may be configured to press against, and exert force on, opposite sides of the inner diameter of a target blood vessel (e.g., aortic wall). The blood-vessel-contact ends 63 can have any suitable shape or form, and advantageously may have an atraumatic (e.g., outwardly-rounded) contact surface.

The structure of the spring implant 60 can mimic that of an elongated compressible spring along the spring lateral dimension d₁ thereof, which may correspond to a major axis of a target blood vessel when the device 60 is used to reshape the target blood vessel to an oval shape. The curved arms 61 that extend to the spring apex 62 can be S-shaped in that they are formed with a curvature that includes one or more inflection points/portions. For example, in the illustrated implementation, each of the curved arms/struts 61 has an inflection point/portion 1101, such that an inner curved portion 1102 has a curvature that opens in a different direction from the curvature of an outer curved portion 1103; the inflection point/portion 1101 separates the curved portions 1102, 1103.

Although rectangular tissue-contact ends 63 are shown, it should be understood that in some implementations, the ends 63 may have curved outer surfaces. For example, the ends 63 may be similar in one or more respects to the ends 53 shown in FIGS. 10-1 and 10-2, wherein such curved surfaces may provide atraumatic contact between the device 60 and the inner wall of the target blood vessel 161. In some implementations, the spring form 60 may be laser-cut from a sheet of metal or plastic, or from a cylinder of similar material.

As demonstrated in FIGS. 10-1 and 10-2, the spring element 60, which represents an example implementation of the spring implant 50 shown in FIGS. 10-1 and 10-2, may push the target blood vessel walls radially outwardly to produce an oval reshaping thereof, wherein as pressure increases in the blood vessel, the blood vessel wall may cause compression of the device 60 in the lengthwise dimension d₁, such as by causing crimping in the spring apex 62 and/or in one or more of the curves 1102, 1103 of the curved spring arms 61. Furthermore, compression of the spring implant 60 may occur at a connection point/area 66 between the spring arms 61 and the tissue-contact ends 63. That is, the spring arms 61 may project from the ends 63 at an angle θ₀ with respect to the transfers dimension d₁ of the implant 60, wherein compression of the implant 60 may reduce the angle θ₀ between the arm 61 and the end 63 at the connection point/area 66.

As blood pressure decreases following systole, the spring element 64 may return to the expanded/elongated state shown in FIG. 11, thereby reverting the target blood vessel segment back to the reshaped oval shape. Therefore, the curved arms 61 and/or apex 62 can facilitate the reshaping of the target blood vessel segment to transition between oval and more-circular configurations, thereby restoring some amount of compliance to the target blood vessel and/or otherwise evening-out blood flow therein.

FIG. 12 shows a vessel-reshaping device 67 comprising a plurality of serially-arranged springs 69a-69n in accordance with one or more examples. The individual springs 69 of the implant 67 may be similar in one or more respects to the spring 64 shown in FIG. 11. The plurality of serially-connected, V-shaped spring-biased elements 69 can be interconnected along common elongated tissue-contact ends 68, wherein the tissue-contact sleds/ends 68 are arranged in a manner such that they extend axially to form a support frame for the spring elements 69. The tissue-contact sides/ends 68 can have a curved form/surface to promote vessel reshaping without tissue trauma. In some implementations, the implant 67 comprises a plurality of spring elements that are welded or otherwise attached together in the serial arrangement shown. Alternatively, the springs 69 and tissue-contact ends/sides 68 may be cut/formed from a common sheet or tube of material, such as Nitinol or other metal/alloy.

The structures of the spring implants of FIGS. 11 and 12 can advantageously provide the functionality and/or attributes of other spring-biased implants, while further providing desirable structural stability during compression thereof. The implant devices 60, 67, due to contacting a target blood vessel in only two portions/areas of the target blood vessel, can serve to reshape the target blood vessel without applying excessive/undesirable stresses against the native tissue, such as compared to fully-circumferential stents, for example. Furthermore, the structure of the implants 60, 67 may be sufficiently minimalistic to avoid blocking blood flow into side branches of the target blood vessel 161, particularly in cases where the side branches are positioned outside of the major-axis wall portions of the target blood vessel.

The length Lₛ of the plural-spring-element implant 67 of FIG. 12 in the lengthwise dimension may be between 1-45 cm, and in the biased oval/diastolic configuration the major axis d₁ may be between 1-4 cm (or larger/smaller depending on the particular anatomy). However, other sizes and/or shapes are also within the scope of this disclosure. Although nine spring elements 69 are shown in FIG. 12, it should be understood that the implant 67 may have any number of serially-arranged spring elements 69, such as any number between 5-10, or 2-4, or more than 10 spring elements.

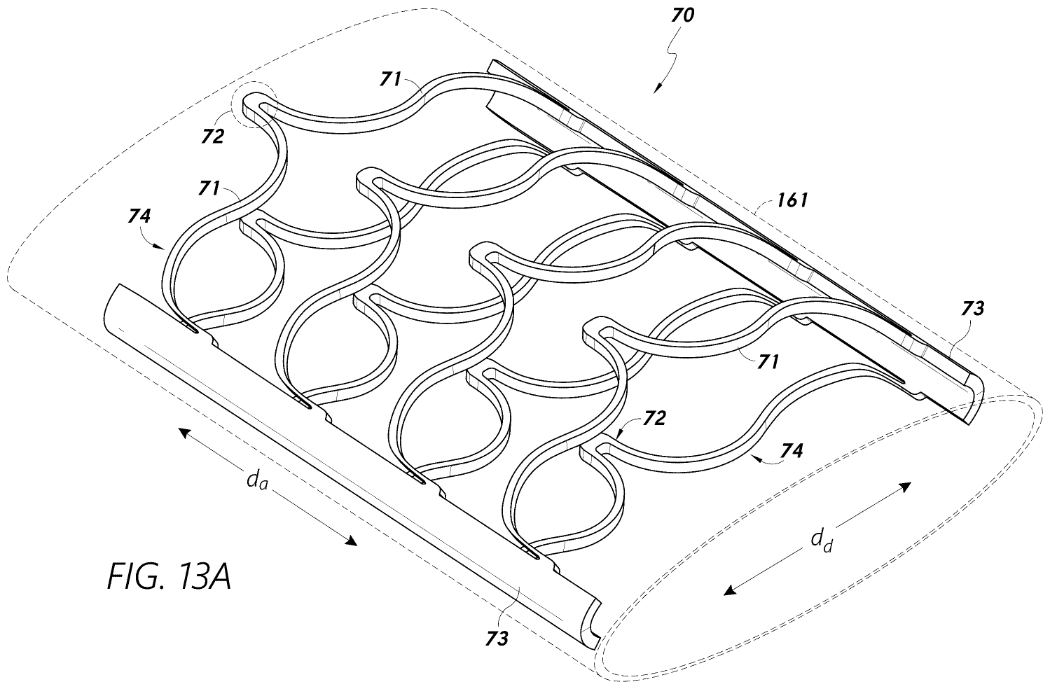

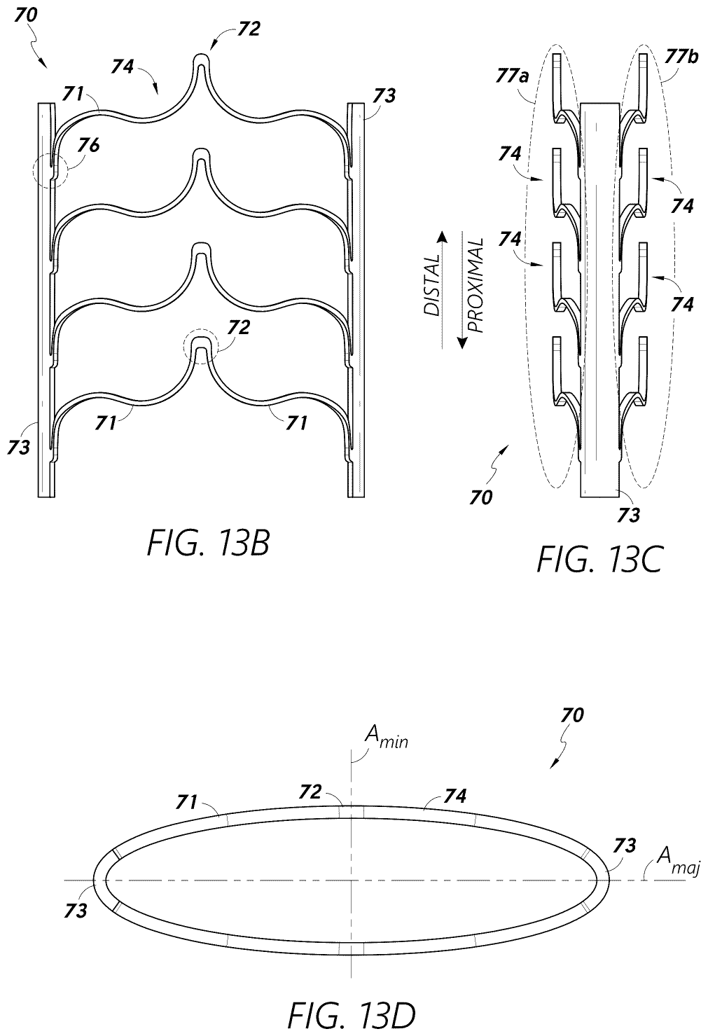

Wishbone/V-shaped blood vessel reshaping implants of the present disclosure may include any suitable or desirable structure or configuration. For example, FIGS. 13A-13D show perspective, side, and axial views, respectively, of a tubular vessel-reshaping spring implant device 70 in accordance with one or more examples. The tubular spring implant device 70 comprises first 77a and second 77b (see FIG. 13C) parallel rows of spring elements 74, wherein the spring elements 74 of the rows 77, together with the tissue-contact sleds/ends 73, form a tubular form as shown in the axial view of FIG. 13D. In such tubular form, the rows 77a, 77b form opposite minor-axis walls of the oval/non-circular stent form shown.

The rows of spring elements can comprise serially-arranged spring elements 74, which may be similar in certain respects to one or more other examples presented herein, such as the examples shown in FIGS. 11 and 12 and described above. For example, the spring elements 74 may be wishbone and/or V-shaped springs which may comprise one or more struts spanning circumferentially between the major-axis ends 73, wherein the spring elements 74 may comprise a spring apex 72 or other feature configured to provide compressibility for the spring element 74 in a manner as to store energy therein when compressed along the diametrical transverse (with respect to the axis of the implant and/or blood vessel) dimension da.

The implant 70 may be cut/formed from a cylinder of metal or other at least partially rigid and/or shape-settable material. For example, the non-circular/oval shape of the implant 70 may be set using heat treatment or other shape-memory-setting treatment. Compared to an implant having only a single row of spring elements as in FIG. 12, the implant 70 may have greater structural stability when implanted within the target blood vessel 161. For example, the implant 70, due to the minor-axis profile/dimension thereof, can be less prone to buckling out-of-plane in an undesirable manner when implanted. That is, due to the circumferential curvature of the spring elements 74, the spring compression of such elements may be constrained by the inner diameter of the target blood vessel, such that the expansion and compression of the spring elements 74 conforms generally to the shape of the blood vessel wall and/or non-circular/oval shape of the shape-set implant, wherein the spring elements 74 may bow radially outwardly to some degree, such that the axial cross-section of the implant 70 shows the spring elements 74 as having outwardly-deflecting curvature, as shown in FIG. 13D. The tissue-contact major-axis ends 73 may likewise have curvature corresponding to the oval cross-sectional shape of the device. When implanted in a target blood vessel 161, the spring elements 74 may generally contact the minor-axis walls of the target blood vessel, or certain gaps made be present between the spring elements 74 and the minor-axis blood vessel walls.

Although each row 77 of spring elements is illustrated as comprising four spring elements, it should be understood that implant devices having parallel rows of spring elements as with the implant 70 may comprise any suitable or desirable number of serially-arranged spring elements, such as two spring elements, one spring element, three spring elements, or more than four spring elements. The spring elements 74 may couple to the tissue-contact sides 73 in any suitable or desirable manner. For example, the struts 71 of the spring elements 74 may project from the ends/sides 73 and deflect at an angle with respect thereto. For example, as in the illustrated implementation, the struts 71 may couple to the ends 73 at a connection area 76 and may deflect in the axial dimension da towards the direction in which the apex 72 points, wherein the struts 71 may follow a curved (e.g., S-curve) path between the connection 76 and the apex 72. In some implementations, the dual-row spring implant device 70 has both major-axis and minor-axis symmetry.

The rows 77a, 77b of spring elements can be coupled to opposite circumferential sides of the tissue-contact ends 73, as shown. The rows 77a, 77b of spring elements can include pairs of corresponding spring elements, wherein each spring element 74 of the first row 77a has a corresponding pair in the second row 77b that is aligned in the longitudinal dimension da.

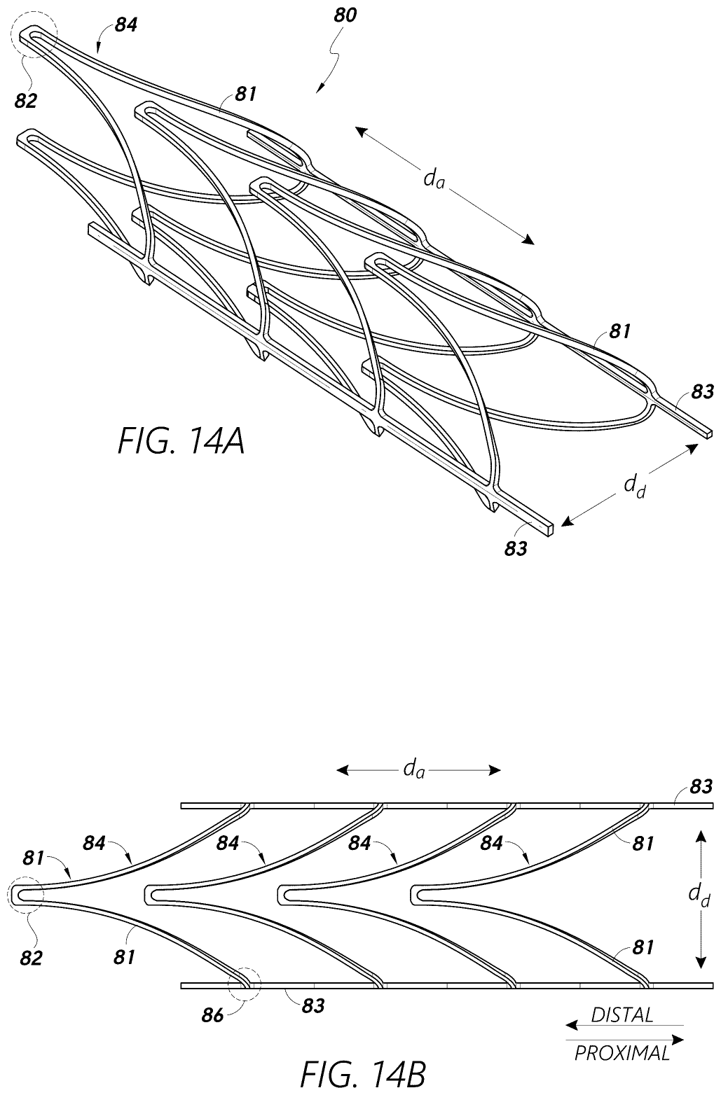

FIGS. 14A and 14B show perspective and side views, respectively, of a tubular vessel-reshaping spring implant device 80 in accordance with one or more examples. The tubular implant 80 includes mirrored rows of spring elements 84, which may be similar in one or more respects to the other examples illustrated and disclosed in connection with the present disclosure. The spring elements 84 may be considered V-shaped and/or wishbone-shaped spring elements/forms in that they comprise struts 81 that project from lateral sides 83 of the implant towards an apex 82, wherein the apex is configured to be compressed such that an angle on a proximal side of the apex can be decreased, thereby storing spring energy in the spring element 84.

Unlike the wishbone spring element 74 described above that comprise arms/struts having curvature inflection between outer ends of the struts and the central apex, the spring elements 84 of FIGS. 14A and 14B may have gradual curvature that does not inflect along a length of the struts 81 between the sides/ends 83 and the apex 82.

The spring elements 84 may form a V-shape having a relatively long axial dimension, wherein axially-adjacent spring elements 84 may be disposed such that an apex of a proximally disposed spring element 84 may axially overlap and/or nest within a void formed by a distally disposed adjacent spring element, thereby providing desirable spring force in a relatively compact axial/longitudinal profile. That is, longitudinally-adjacent spring elements 84 in a given row may be longitudinally-overlapping, wherein an apex of one of the spring elements is disposed/nested within a space formed by an interior angle of an apex of the other spring element.

At side connections 86, the struts 81 of the spring elements 84 may project/jut away from the tissue-contact ends 83, which may have any shape or form as described herein, at a distally-deflected angle relative to the axially-aligned ends 83. That is, as illustrated, the struts 81 may project circumferentially/laterally from the ends 83 and curve/deflect distally towards a distally-oriented apex 82. The implant 80 may have an oval and/or circular axial cross-sectional shape.

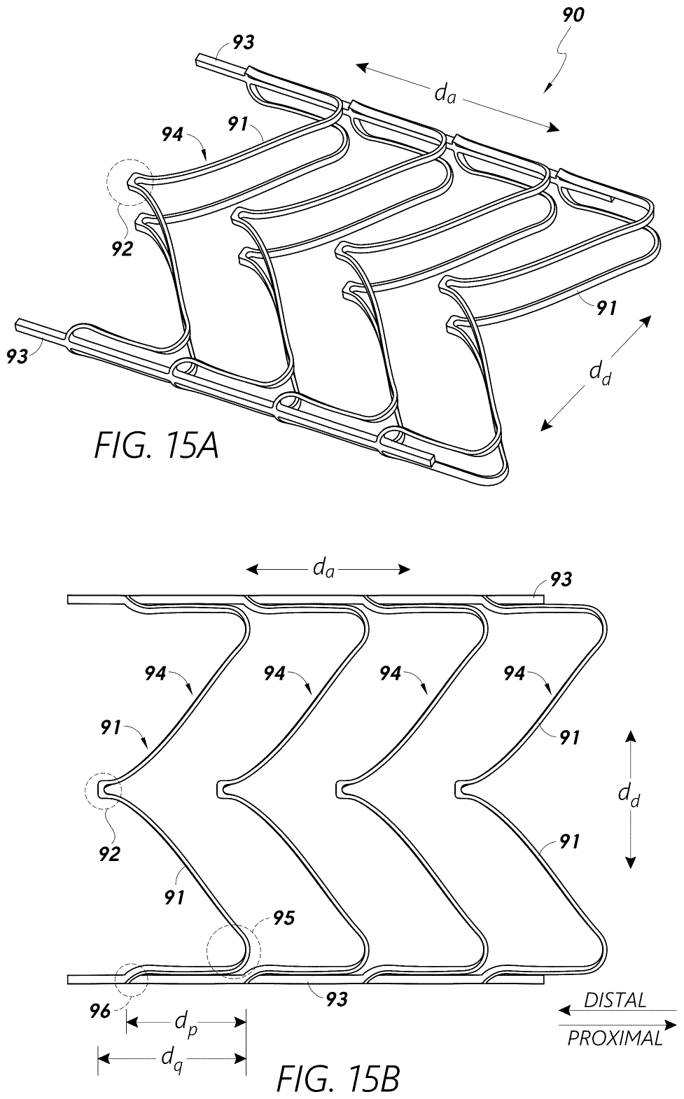

FIGS. 15A and 15B show perspective and side views, respectively, of a tubular vessel-reshaping spring implant device in accordance with one or more examples. The spring elements 94 of the implant 90, similarly to certain other examples presented herein, comprise compressible apices 92 configured to store spring energy for vessel reshaping as described in detail herein. The spring implant device 90 includes one or two (as illustrated) rows of spring elements 94, wherein each row comprises one or more spring elements disposed in serially-arranged rows of spring elements, as shown.

The spring elements 94 may differ in one or more respects relative to spring elements described above. For example, the spring elements may comprise struts 91 projecting from tissue-contact ends/sides 93, wherein the struts 91 are deflected first proximally moving away from the ends 93 at or near a coupling connection 96 with the end 93, wherein the struts may include an inflection of curvature allowing for the strut to deflect back distally towards the apex 92. For example, in the illustrated implementation, the struts 91 deflect proximally from the ends 93 a distance de before curving back to deflect distally towards the apex 92, wherein the distal projecting portion of the strut 91 may span an axial distance de, as illustrated. At least a portion of the proximally-deflected portion of the strut may align in parallel with the sides 83, as shown.

The struts 91 may be considered crooked struts having both proximal and distal deflection. The curving of the struts proximally and distally can form a bend 95 feature, which may provide curvature configured to allow for compression of the spring element 94 in the lateral/circumferential dimension da. That is, the spring elements 94 may store spring energy in a plurality of bends of the spring element, including the apex 92, the bends 95, and/or the connection bends 96, which may advantageously allow for substantial compression and expansion in the diametrical/lateral dimension da of the spring elements 94.

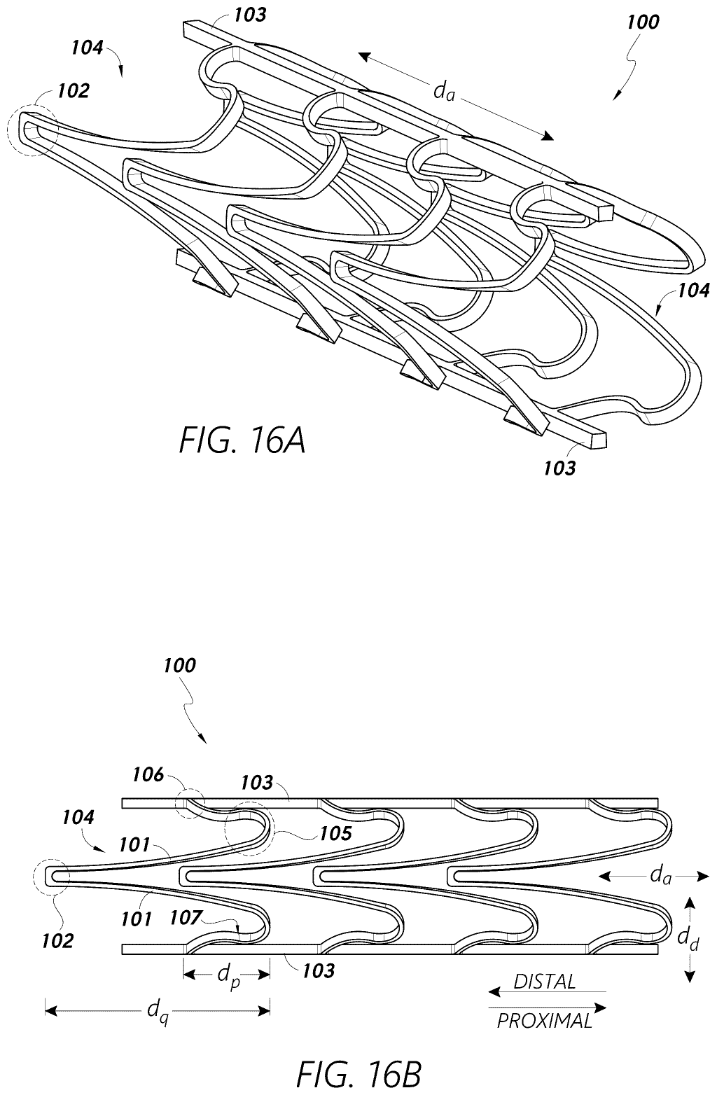

FIGS. 16A and 16B show perspective and side views, respectively, of a tubular vessel-reshaping spring implant device 100 in accordance with one or more examples. The spring elements 104 of the implant 100, similarly to certain other examples presented herein, comprise compressible apices 102 configured to store spring energy for vessel reshaping as described in detail herein. The spring implant device 100 includes one or two (as illustrated) rows of spring elements 104, wherein each row comprises one or more spring elements arranged in a serial fashion, as shown.

The spring elements 104 may comprise struts 101 projecting from tissue-contact ends/sides 103, wherein the struts 101 are deflected first proximally moving away from the ends 103 at or near a coupling connection 106 with the respective end 103. The struts may include an inflection point/portion 107 allowing for the strut 101 to deflect back distally towards the apex 102. For example, in the illustrated implementation, the struts 101 deflect proximally from the ends 103 a distance de before curving back to deflect distally towards the apex 102, wherein the distal projecting portion of the strut 101 may span an axial distance da, as illustrated. Therefore, the struts 101 may be considered crooked struts having both proximal and distal deflection. The curving of the struts 101 proximally and distally can form a proximally-oriented lobe 105 feature, which may provide curvature configured to allow for compression of the spring element 104 in the lateral/circumferential dimension da. That is, the spring elements 104 may store spring energy in a plurality of bends of the spring element, including the apex 102, the lobes 105, and/or the connection bends 106, which may advantageously allow for substantial compression and expansion in the diametrical/lateral dimension da of the spring elements 104.

As with the example of FIGS. 14A and 14B, the spring elements 104 may axially/longitudinally overlap to some degree, wherein an apex of a proximally positioned spring element may project into and/or nest within a void/contour formed by a V-shaped apical form of a distally-positioned adjacent strut in the same row, as shown.

### Corrugated Spring Stents

Various types of spring implant devices are disclosed above, some of which include frames and/or struts configured to bend in a spring-like manner, wherein such bends are generally in a plane of the implant device. That is, with respect to wishbone spring elements, such spring elements may lie in a flat or at least partially curved (e.g., cylinder arc/portion) plane, wherein the bend(s) of such spring elements generally bend within the plane of the spring element. In some implementations, vessel-reshaping spring implant devices of the present disclosure can comprise circumferentially-corrugated stent frames/structures, wherein spring force/energy is stored in the bends of the corrugations of the stent frame.

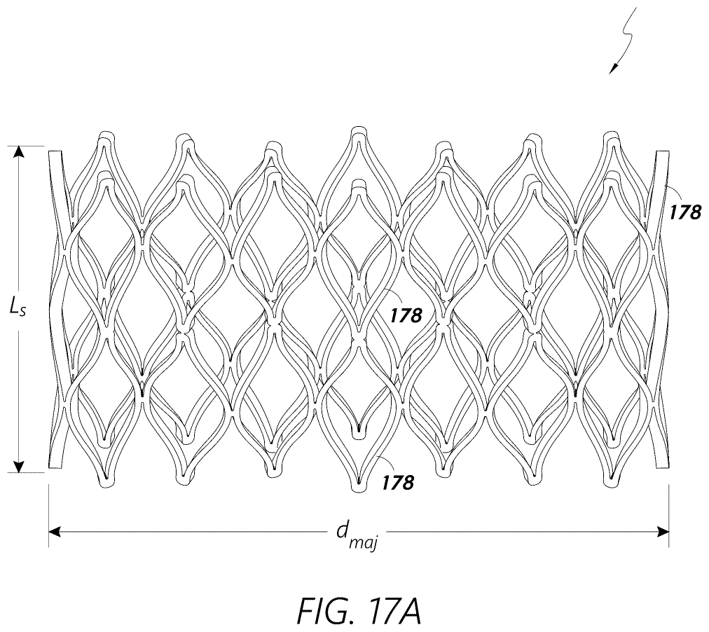

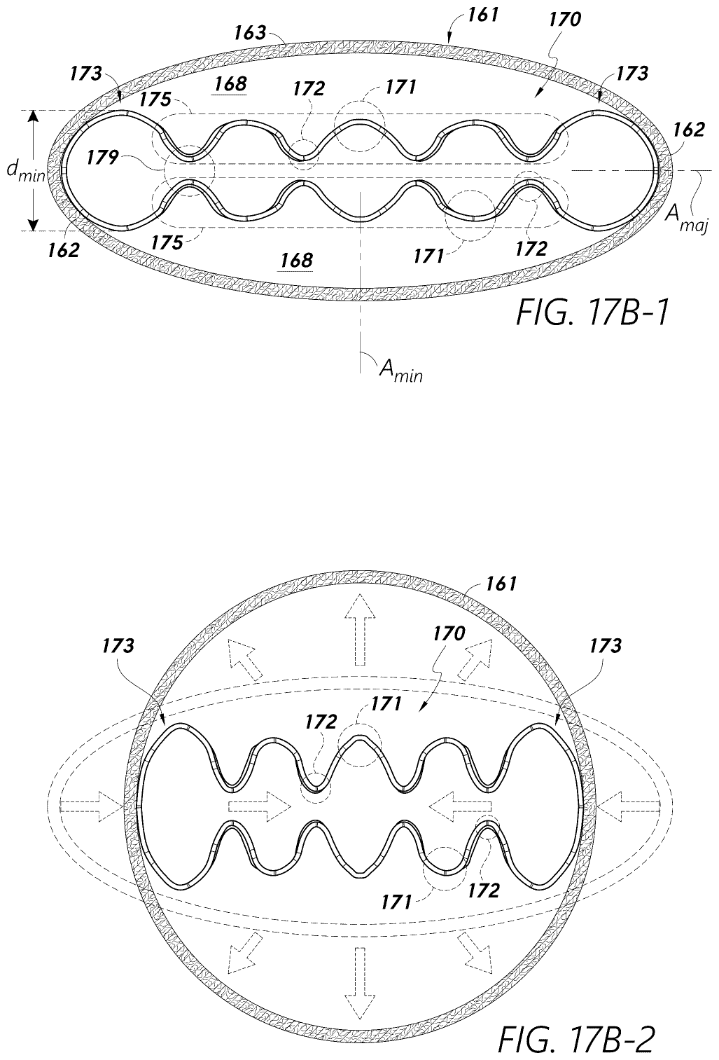

FIG. 17A shows a perspective view of a compressible corrugated spring stent 170 in accordance with one or more examples. FIGS. 17B-1 and 17B-2 show the compressible spring stent 170 in expanded and compressed configurations, respectively, within a blood vessel in accordance with one or more examples. The stent 170 may comprise a stent frame formed of a plurality of struts 178 arranged in an open-cell pattern, or any other arrangement suitable for a stent implant. The open cells of the stent frame may have a diamond-type shape or any other shape. FIG. 17A shows a perspective view showing the stent 170 having a length Lₛ, which may be any suitable or desirable length, such as a length between 2-10 cm. The major-axis dimension dₘₐⱼ of the stent 170 may be designed to produce ovalized stretching in a target blood vessel segment as described in detail herein.

The spring-biased stent element 170 can comprise a central spring-like structure configured to be disposed within an insufficiently-compliant section of a target blood vessel, such as the aorta, with opposite ends 173 of the spring stent pressed against the blood vessel wall to force the blood vessel wall to assume an oval-shaped configuration, for example during diastole. For example, the spring stent 170 may have an elongated transverse dimension dₘₐⱼ with respect to the axis of the target blood vessel, which is advantageously greater than a natural diameter of the target blood vessel, whereas the stent dimension dₘᵢₙ orthogonal to the long dimension dₘₐⱼ may be less than the natural diameter of the target blood vessel.

With respect to the axial view of FIG. 17B-1, the stent 170 may have an elongated/stretched-out shape, such that the major-axis dimension dₘₐⱼ of the stent is substantially greater than the maximum minor-axis dimension dₘᵢₙ. With the stent 170 deployed within the target blood vessel 161, the major-axis ends 173 of the stent may push the sidewalls 162 of the blood vessel 161 radially outwardly to produce an oval shape or other non-circular shape in the blood vessel. The stent 170, when deployed in low-pressure conditions, can be oriented in-line with a long axis of the oval-shaped blood vessel segment, wherein contact portions 173 at opposite/both ends of the stent 170 are configured to press against the inner blood vessel wall.

As shown in the axial view of FIG. 17B-1, the walls of the stent 170 may have a corrugated, scalloped, and/or wavy form, such that the stent frame forms outwardly convex 171 and concave 172 surfaces/portions. That is, the curvature of the corrugated walls of the stent form bends 171, 172 that have a shape-memory such that compression of such bends in the major axis dimension dₘₐⱼ stores energy in the form of a spring force in favor of elongation of the stent in the major axis dimension.

The corrugations 171, 172 in the stent frame may be primarily associated with minor-axis walls 175, wherein the minor-axis walls 175 may span between tissue-contact end walls/portions 173, which may have the form of curved lobes/bulges, as illustrated. The curvature of the ends 173 may provide atraumatic tissue contact surfaces, as described in detail herein, and may further serve to shape the blood vessel walls by producing a curvature in the blood vessel walls 162 that facilitates a desirable oval shape in the blood vessel cross-section. The spring-like corrugated walls 175 can extend, together with the end segments 173, along the entire length of the major-axis of the blood vessel, as shown. The corrugations 171, 172 may effectively produce a circumferential spring in the stent frame itself extending between the curved ends/edges 173 that presses against the blood vessel wall. The generally wavy formation of the stent walls 175, in order to produce the desired spring functionality, can be designed to have a spring constant sufficient to impart the desired blood vessel reshaping.

FIG. 17B-1 shows the stent 170 in a relaxed/biased laterally-expanded configuration that is elongated with respect to the major-axis dimension dₘₐⱼ to a biased shape thereof, wherein in the expanded configuration shown in FIG. 17B-1, the stent 170 is configured to produce a non-circular/oval shape in the target blood vessel in which it is deployed. As the luminal blood pressure increases, the pressure forces within the blood vessel may produce hoop stress on the blood vessel walls that promotes circularization of the blood vessel with sufficient force to overcome the spring biasing of the shape-memory of the stent frame 170, thereby causing the bends 171, 172 in the minor-axis walls 175 to compress, reducing the major-axis dimension dₘₐⱼ of the stent 170 and storing spring energy in the stent frame. Once the high pressure levels dissipate sufficiently, such as in connection with diastole, the shape memory forces of the stent may permit the stent to expand/elongate in the major-axis dimension dₘₐⱼ to thereby reshape the blood vessel to the oval shape shown in FIG. 17B-1.

The thickness, configuration, and/or other attribute(s) of the stent frame 170 can be designed to produce a desired spring constant in the implant device 170. For example, the spring constant may be designed such that during diastole the end portions 173 is pressed against the arterial wall, forcing it to assume the oval-shaped configuration shown in FIG. 17B-2. The elevated pressures associated with systole, acting against the aortic wall, can overcome the springs forces and allow the aortic section to assume a more circular shape, thereby compressing the spring as in FIG. 17B-2. As the pressure is once again lowered, such as during diastole, the spring 170 may revert to its extended state, reverting the target blood vessel segment back to the oval shape. Therefore, the spring stent 170 can allow the target blood vessel segment to transition between oval and more-circular configurations, thereby restoring some amount of compliance to the otherwise insufficiently-compliant blood vessel.

A corrugated spring stent 170 can advantageously produce desired blood vessel reshaping without substantially interfering with blood flow through the target blood vessel, and further with reduced contact with the inner diameter of the blood vessel, thereby potentially reducing the impact on the native anatomy during the reshaping process. For example, the contact between the stent 170 and the blood vessel inner diameter may be primarily in the area of the major-axis wall portions 162. Generally, gaps 168 may be present between the minor-axis spring walls 175 and the minor-axis blood vessel walls 163, wherein all or a portion of the spring walls 175 may not contact the blood vessel walls 163 when implanted, at least with respect to certain phases/portions of the cardiac cycle. Therefore, the risk of irritation to large portions of the blood vessel wall and/or the risk of producing thrombosis may be reduced compared to stents that have full-circumferential contact between the stent frame and the blood vessel wall. The undulating sidewalls 175, in the relaxed state of the stent 170, can be generally parallel to one another, as shown. The convex 171 and concave 172 bends in the walls 175 form peaks and valleys, respectively, from the outer perspective. The end portions 173 can corm circumferentially-open lobes/tubes, wherein the openings 179 thereof couple to the sidewalls 175.

In some implementations, the stent frame 170 is a bare-metal, wherein the frame is not covered internally or externally by a fluid-tight covering. Therefore, the origin of the driving force required to transition the frame 170 from the oval shape to the more circular shape may necessarily be based on hoop pressure/stress against the vessel inner diameter. In some implementations, the stent 170 includes a covering, which can comprise any type of biocompatible material, such as, but not limited to, expanded polytetrafluoroethylene (ePTFE), polyester, polyurethane, fluoropolymers (e.g., perfouorelastomers and the like), polytetrafluoroethylene, silicones, urethanes, ultra-high molecular weight polyethylene, aramid fibers, and combinations thereof.

### Dual-Spring Vessel-Reshaping Implants

Some examples are presented herein wherein spring-type vessel-reshaping implant devices comprise spring elements configured to compress in a major-axis dimension and/or along a circumferential arc/segment that aligns generally with the major-axis dimension of the implant device. However, it should be understood that examples of the present disclosure may be configured to compress and multiple dimensions/planes in connection with some examples.

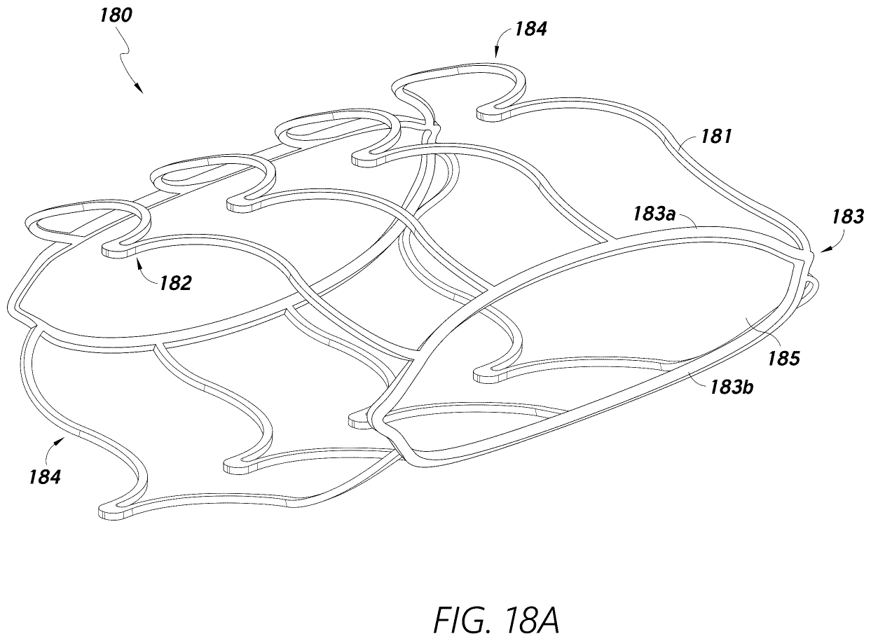

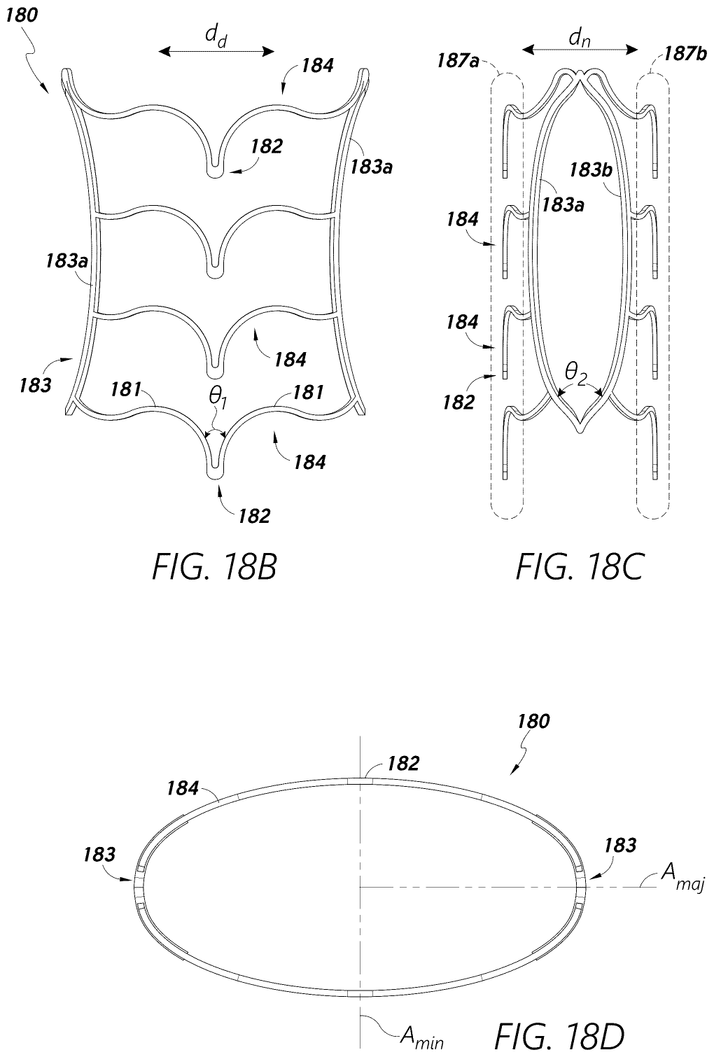

FIGS. 18A-18D show perspective, side, and axial views, respectively of a vessel-reshaping implant device configured to compress in orthogonal planes in an expanded configuration in accordance with one or more examples. The spring implant 180 may include a plurality of lateral spring elements 184, which may be similar in any respect to any of the spring examples disclosed herein. For example, the spring elements 184 may comprise wishbone/V-shaped springs including central apices configured to bend/compress in a manner as to effect a spring compression. The spring elements 184, unlike other examples disclosed herein, may be coupled at lateral ends thereof to transverse spring elements 183 configured to compress in a minor-axis dimension Amin of the implant device 180. Therefore, the spring implant 180 may be configured to compress in both a major-axis dimension Aₘₐⱼ and a minor-axis dimension Amin, which may advantageously provide for substantial compression and/or re-shaping functionality for the implant 180. That is, the spring elements 184, rather than converging on the sides of the implant at linear bars or sleds, the struts 181 of the spring elements 184 may be disposed between compressible elliptical supports 183. The side springs 183 may have a shape of an ellipse, leaf, or similar shape. For example, the springs 183 may have the shape of an ellipse with pointed ends, as shown. The spring struts 183 can form an open cell 185, as shown.

The transverse compression elements 183 may be implanted on both major-axis ends/sides of the implant 180. Furthermore, the transverse compression elements 183 may comprise one or more struts, which may be arranged in a generally-elliptical arrangement, wherein transverse compression of the springs 183 in the minor-axis dimension Amin can serve to reduce an angle θ₁ at connecting ends of the struts 183a, 183b. The combination of compression and expansion of the spring elements 184 and spring elements 183 can facilitate transition of the target blood vessel wall between elliptical/oval and more-circular shapes by producing bi-planar compression, in which such plans of compression may be generally orthogonal to one another. Although the compression of the spring elements 184 is described as being aligned with the major axis Aₘₐⱼ of the implant 180, it should be understood that the spring elements 184 may compress in a plane that conforms to the circumferential curvature of the implant. That is, the compression of the spring elements 184 may be generally a circumferential curve/arc that, while generally aligned with the major axis Aₘₐⱼ, is not a flat parallel plane with the major axis Aₘₐⱼ, but generally aligns therewith. Furthermore, the curvature of the end springs 183 may likewise be in a plane that curves along with the circumference of the major-axis end portions of the implant's oval shape. However, the compression of such springs 183, even where not exactly parallel with the minor axis Amin, may be described as generally aligned with the minor axis Amin to illustrate the multi-dimensional compression of the implant device 180.

The implant 180 includes two rows of spring elements 184, as with other examples disclosed herein, wherein the rows 187a, 187b are connected to opposite sides of the elliptical side springs 183, forming a structure that may resemble in one or more respects dual-plate/plane spring. As configured, the two planes of compression of the implant 180 can be approximated to each other and distanced from each other, thereby facilitating the transition of the aortic cross-sectional shape between oval and more-circular configurations. The primary dimension of compression of the spring elements 184 may be considered a first transverse dimension da, or “lateral” dimension, whereas the primary dimension of compression of the side springs 183 may be considered a second transverse dimension dₙ, or ‘vertical’ dimension.

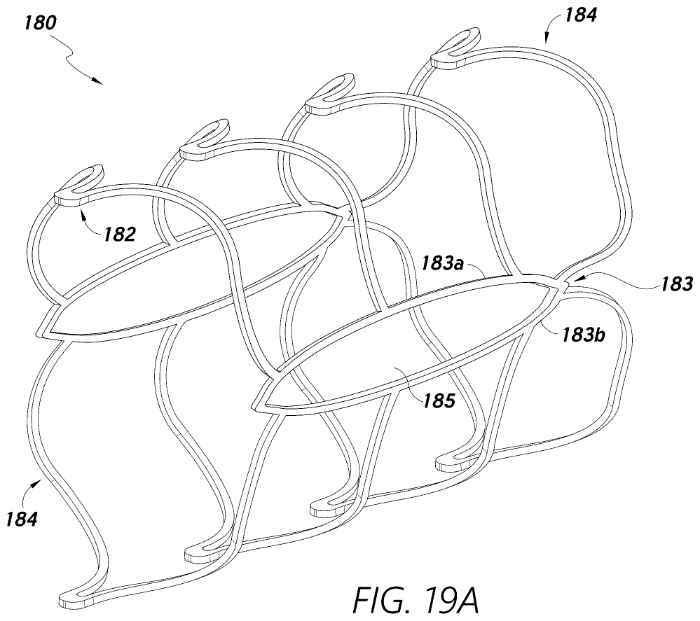

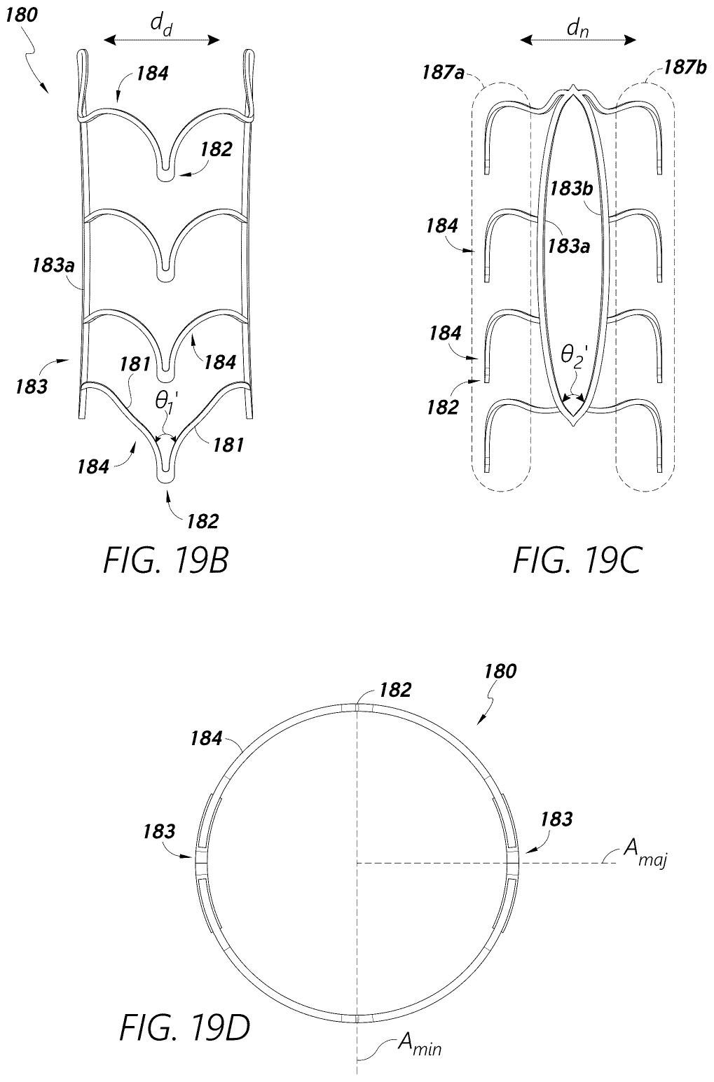

The transverse spring struts 183a, 183b on both major-axis sides of the implant can be configured to be pressed against the inner blood vessel wall and transition between compressed and expanded configurations. FIGS. 19A-19D show perspective, side, and axial views, respectively of the vessel-reshaping implant device 180 of FIGS. 18A-18D in a compressed configuration in accordance with one or more examples. As the side springs 183 are compressible in a direction that is generally/approximately orthogonal to the plane of the lateral spring bends 182, the implant 180 can advantageously facilitate reshaping of the target blood vessel wall in two perpendicular planes, namely the plane of the compressible spring apex 182 and the plane of the compressible elliptical side supports 183.

When both the lateral spring elements 184 and the side springs 183 are compressed, the axial cross-sectional shape of the implant 180, as shown in FIG. 19D, may become more circular. For example, such circularized shape may form in the implant 180 in response to increased blood pressure compressing the frame of the implant to conform to a more circularized shape of the target blood vessel. In the compressed configuration, the angle separating the struts 181 at the apices 182 of the V-shaped/wishbone spring elements 184 may be decreased from a relatively greater angle θ₁ to a lesser angle θ₁. Furthermore, the angle θ₂ of the side springs struts 183 may be compressed to a compressed smaller angle θ₂ during systole or other high-pressure condition.

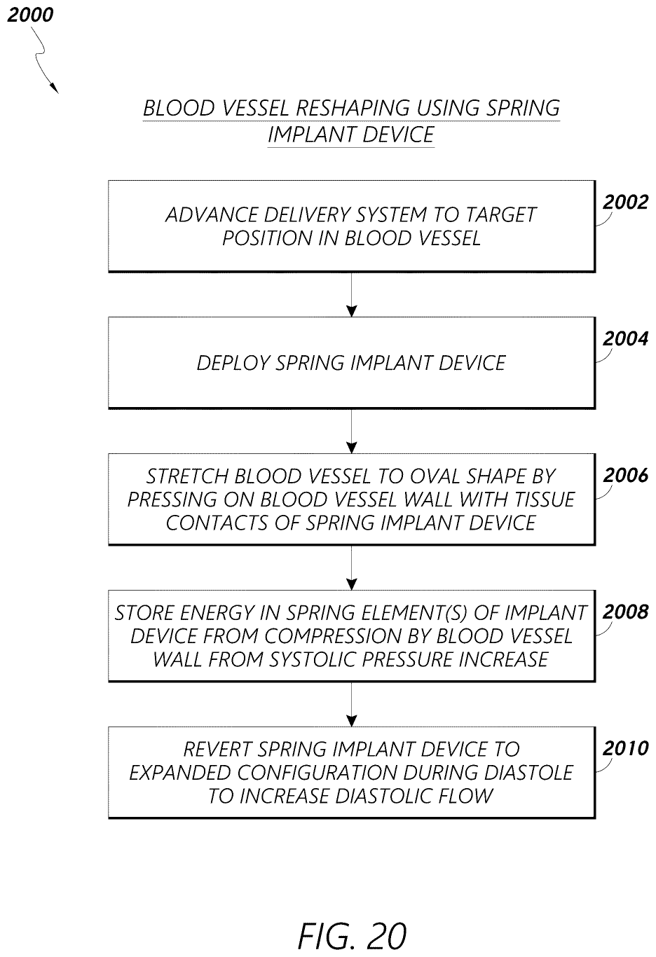

FIG. 20 is a flow diagram illustrating a process for reshaping a blood vessel using a compressible implant device in accordance with one or more examples. At block 2002, the process 2000 involves advancing a delivery system to a target position in a blood vessel, such as the aorta. For example, the delivery system may be advanced through a percutaneous introducer or other minimally-invasive access into the vasculature of the patient, and further within the vasculature to a target position within the aorta of the patient. The delivery system may include one or more catheters/sheaths and/or a nosecone or other feature configured to facilitate the forward advancement of the delivery system through tortuous anatomy of the vasculature.

At block 2004, the process 2000 involves deploying a spring implant device comprising one or more spring elements, such as a tubular and/or stent implant device as disclosed in detail herein, from the delivery system. The spring implant may be transported to the target site in a radially crimped configuration to fit within a catheter/sheath having relatively small profile. The spring implant may have a relaxed shape that resembles an oval or other non-circular shape, wherein the implant has a relatively long lateral dimension relative to the natural diameter of the target blood vessel segment.

At block 2006, the process 2000 involves ovalizing the target blood vessel by pressing against opposite sides of the inner wall of the blood vessel with tissue-contact ends/sides of the spring implant device at two tissue-contact areas, as described in detail herein. The ovalizing of the target blood vessel may be performed during a state of relatively-low blood pressure in the blood vessel lumen, which may allow the spring implant device to assume a laterally-stretched/expanded shape associated with a biased/relaxed shape thereof.

At block 2008, the process 2000 involves compressing, or allowing compression of, the spring implant by the blood vessel wall in response to increased luminal pressure, which may increase hoop stress in the blood vessel wall sufficient to overcome the spring biasing of the implant. The implant may thereby become at least partially compressed in a lateral dimension thereof, which may cause spring energy to be stored in the spring element(s) of the implant, which may comprise any of the spring features disclosed herein. As the implant is compressed, the blood vessel may assume a more-circular shape, which can increase a volume thereof.

At block 2010, the process 2000 involves reverting the spring implant device to the expanded configuration thereof as pressure levels decrease, such as in connection with the transition from systole back to diastole, thereby reverting the blood vessel segment to the non-circular/oval cross-sectional shape. Such transition can effectively decrease the volume of the blood vessel segment, thereby increasing pressure and flow therein to increase diastolic perfusion in a manner consistent with healthy, compliant blood vessels.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.

Example 1: An implant device comprising a circumferentially-open stent frame having a non-circular axial cross-sectional shape, the stent frame forming a circumferential gap in a relaxed configuration of the stent frame.

Example 2: The implant device of any example herein, in particular example 1, wherein the stent frame includes first and second circumferential ends that define the circumferential gap.

Example 3: The implant device of any example herein, in particular example 2, wherein each of the first and second circumferential ends comprises an inwardly-deflected free edge of the stent frame.

Example 4: The implant device of any example herein, in particular example 3, wherein each of the inwardly-deflected free ends forms a curved contact surface.

Example 5: The implant device of any example herein, in particular any of examples 1-4, wherein the stent frame, in the relaxed configuration, forms first and second circumferential bends.

Example 6: The implant device of any example herein, in particular example 5, wherein the first and second bends are configured to store spring energy when an internal angle thereof is increased.

Example 7: The implant device of any example herein, in particular example 5 or example 6, wherein the first and second bends are positioned on opposite circumferential sides of the stent frame.

Example 8: The implant device of any example herein, in particular example 7, wherein the stent frame further forms a third circumferential bend positioned opposite the circumferential gap.

Example 9: The implant device of any example herein, in particular any of examples 5-8, wherein the first and second bends define a major-axis diameter of the stent frame.

Example 10: The implant device of any example herein, in particular any of examples 1-9, wherein the stent frame has an open diamond cross-sectional shape.

Example 11: The implant device of any example herein, in particular any of examples 1-10, wherein the stent frame comprises shape-memory material shape-set to the non-circular cross-sectional shape.

Example 12: The implant device of any example herein, in particular any of examples 1-11, further comprising an outer covering that spans the circumferential gap of the stent frame.

Example 13: An implant device comprising a planar body formed into a curved back portion, first and second elbow bends on opposite sides of the back portion, and first and second arm portions extending from the first and second elbow bends, respectively, the first and second arm portions having free edges.

Example 14: The implant device of any example herein, in particular example 13, wherein the planar body comprises a stent frame including struts forming open cells.

Example 15: The implant device of any example herein, in particular example 13 or example 14, wherein the planar body is spring-biased, such that the first and second elbow bends are configured to store spring energy when an angle thereof is forcibly changed from a relaxed configuration thereof.

Example 16: The implant device of any example herein, in particular any of examples 13-15, wherein a fluid-tight covering at least partially covers at least one of an internal diameter or an external diameter of the planar body.

Example 17: The implant device of any example herein, in particular example 16, wherein the covering spans a gap between the free edge of the first arm portion and the free edge of the second arm portion.

Example 18: The implant device of any example herein, in particular any of examples 13-17, wherein the curved back portion has a central bend that is parallel with the first and second elbow bends.

Example 19: The implant device of any example herein, in particular any of examples 13-18, wherein the first and second elbow bends of the planar body hold the first and second arm portions, respectively, at spring-set angles relative to the back portion.

Example 20: The implant device of any example herein, in particular any of examples 13-19, wherein the first and second elbow bends define a longest diameter of the planar body.

Example 21: A method of managing flow in a blood vessel, the method comprising deploying a C-shaped stent in circumferential contact with a target blood vessel, the C-shaped stent having a non-circular axial cross-sectional shape and forming a circumferential gap, and reshaping the target blood vessel to an oval cross-sectional shape using the stent.

Example 22: The method of any example herein, in particular example 21, further comprising advancing a delivery system to a target site in the target blood vessel, wherein said deploying the stent occurs within the target blood vessel at the target site.

Example 23: The method of any example herein, in particular example 21 or example 22, further comprising aligning the circumferential gap with an ostium of a side branch of the target blood vessel.

Example 24: The method of any example herein, in particular any of examples 21-23, wherein said deploying the stent involves placing the stent on an outer surface of the target blood vessel.

Example 25: The method of any example herein, in particular example 24, wherein the C-shaped stent includes first and second free circumferential edges that are curved away from the outer surface of the target blood vessel.

Example 26: The method of any example herein, in particular any of examples 21-25, wherein said reshaping of the target blood vessel involves ovalizing the target blood vessel using a major-axis dimension of the stent.

Example 27: The method of any example herein, in particular any of examples 21-26, wherein said reshaping of the target blood vessel involves pressing against opposite circumferential portions of the blood vessel with major-axis bends of the stent.

Example 28: The method of any example herein, in particular any of examples 21-27, further comprising allowing free circumferential contact surfaces of the stent to come into contact with one another in response to elevation in blood pressure within the target blood vessel.

Example 29: The method of any example herein, in particular example 28, further comprising, after the contact surfaces have come into contact, allowing the blood vessel to circularize the stent by applying radially-inward force on major-axis tissue-contact portions of the stent.

Example 30: The method of any example herein, in particular example 29, further comprising, after said circularizing of the stent, reshaping the target blood vessel back to the oval cross-sectional shape using shape memory of the stent configured to expand a major-axis of the stent.

Example 31: The method of any example herein, in particular any of examples 21-30, wherein said reshaping the target blood vessel increases diastolic blood pressure in at least a portion of the target blood vessel.

Example 32: An intravascular spring implant device comprising first and second tissue-contact end portions configured to press against an inner diameter of a target blood vessel, and one or more spring elements configured to be compressed in a manner as to reduce a distance between the first and second tissue-contact end portions.

Example 33: The intravascular spring implant device of any example herein, in particular example 32, wherein each of the one or more spring elements comprises a V-shaped spring.

Example 34: The intravascular spring implant device of any example herein, in particular example 33, wherein the V-shaped spring has a wishbone form.

Example 35: The intravascular spring implant device of any example herein, in particular example 33 or example 34, wherein the V-shaped spring forms a central apex connected to the first and second tissue-contact end portions by respective struts.

Example 36: The intravascular spring implant device of any example herein, in particular example 35, wherein the struts are curved.

Example 37: The intravascular spring implant device of any example herein, in particular example 36, wherein the struts have first and second curved portions separated by an inflection portion.

Example 38: The intravascular spring implant device of any example herein, in particular example 37, wherein the struts project from one of the first or second tissue-contact end portions, deflect in a first longitudinal direction on a first side of the inflection portion, and project in a second longitudinal direction opposite the first longitudinal direction on a second side of the inflection portion towards an apex spring feature that points in the second longitudinal direction.

Example 39: The intravascular spring implant device of any example herein, in particular any of examples 36-38, wherein the struts have an S-shape.

Example 40: The intravascular spring implant device of any example herein, in particular any of examples 32-39, wherein the one or more spring elements comprises a plurality of serially-arranged spring elements.

Example 41: The intravascular spring implant device of any example herein, in particular example 40, wherein apices of the plurality of spring elements are aligned along a lengthwise dimension of the implant device.

Example 42: The intravascular spring implant device of any example herein, in particular example 41, wherein the plurality of spring elements are each coupled at opposite ends thereof to the first and second tissue-contact end portions, respectively.

Example 43: The intravascular spring implant device of any example herein, in particular any of examples 40-42, wherein the plurality of serially-arranged spring elements comprises first and second rows of spring elements.

Example 44: The intravascular spring implant device of any example herein, in particular example 43, wherein each spring element of the first row of spring elements is longitudinally-aligned with a corresponding spring element of the second row of spring elements.

Example 45: The intravascular spring implant device of any example herein, in particular example 43 or example 44, wherein the first row of spring elements is coupled to a first side of the first and second tissue-contact end portions and the second row of spring elements is coupled to a second side of the first and second tissue-contact end portions.

Example 46: The intravascular spring implant device of any example herein, in particular any of examples 43-45, wherein the one or more spring elements are configured to compress primarily in a first transverse dimension with respect to an axis of the spring implant device, and the first and second tissue-contact end portions comprise first and second end spring elements, respectively, configured to compress primarily in a second transverse dimension that is orthogonal to the first transverse dimension.

Example 47: The intravascular spring implant device of any example herein, in particular example 46, wherein the first and second end spring elements have an open-cell oval shape.

Example 48: The intravascular spring implant device of any example herein, in particular example 47, wherein the first and second end spring elements each comprise a first strut coupled to the first row of spring elements and a second strut coupled to the second row of spring elements.

Example 49: The intravascular spring implant device of any example herein, in particular example 48, wherein the first strut and the second strut form diametrically-opposite sides of an oval spring form.

Example 50: The intravascular spring implant device of any example herein, in particular any of examples 43-49, wherein the implant device has an oval axial cross-sectional shape.

Example 51: The intravascular spring implant device of any example herein, in particular any of examples 43-50, wherein the first and second tissue-contact end portions and the one or more spring elements are cut from a metal tube.

Example 52: The intravascular spring implant device of any example herein, in particular any of examples 40-51, wherein adjacent pairs of the plurality of spring elements are longitudinally overlapping.

Example 53: The intravascular spring implant device of any example herein, in particular example 52, wherein each of the plurality of spring elements comprises an apex, and a longitudinally-adjacent pair of the plurality of spring elements comprises a first spring element positioned such that an apex of the first spring element is disposed in a space formed by an interior angle of an apex of a second spring element of the longitudinally-adjacent pair of the plurality of spring elements.

Example 54: The intravascular spring implant device of any example herein, in particular any of examples 32-53, wherein the first and second tissue-contact portions each comprise a curved contact surface that curves radially outwardly.

Example 55: The intravascular spring implant device of any example herein, in particular any of examples 32-54, further comprising a stent frame, wherein the stent frame forms the first and second tissue-contact end portions and the one or more spring elements.

Example 56: The intravascular spring implant device of any example herein, in particular example 55, wherein the stent frame comprises a plurality of rows of open cells formed by struts.

Example 57: The intravascular spring implant device of any example herein, in particular example 55 or example 56, wherein the one or more spring elements comprise first and second undulating sidewalls of the stent frame.

Example 58: The intravascular spring implant device of any example herein, in particular example 57, wherein the first and second sidewalls are parallel in a relaxed state of the implant device.

Example 59: The intravascular spring implant device of any example herein, in particular example 57 or example 58, wherein undulations of the first and second sidewalls comprise convex and concave bends that are configured to store spring energy when the first and second sidewalls are compressed with respect to a major-axis dimension of the stent frame.

Example 60: The intravascular spring implant device of any example herein, in particular any of examples 57-59, wherein the first and second tissue-contact end portions comprise circumferentially-open tubes providing an outwardly-convex tissue-contact surface.

Example 61: The intravascular spring implant device of any example herein, in particular example 60, wherein the first and second sidewalls emanate from open sides of the first and second tissue-contact end portions.

Example 62: The intravascular spring implant device of any example herein, in particular any of examples 57-61, wherein the first and second undulating sidewalls include at least three apices.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An intravascular compliance-enhancing spring implant device comprising:
a cylindrical tubular frame defining a longitudinal axis and a non-circular cross-sectional shape having a major axis diameter and a minor axis diameter that is less than the major axis diameter, the tubular frame constructed for cyclically altering a shape of a target blood vessel;
first and second longitudinal tissue-contact rails associated with first and second major-axis ends, respectively, of the tubular frame and adapted to press against an inner diameter of a target blood vessel in an atraumatic manner, wherein a distance between the first and second longitudinal tissue-contact rails is adjustable; and
first and second lateral connecting strut arrays associated with minor-axis sides of the tubular frame, the first and second lateral connecting strut arrays spanning between the first and second tissue-contact rails on diametrically opposite sides of a lumen of the tubular frame and adapted to be compressed along a major-axis dimension of the tubular frame in a manner as to reduce the distance between the first and second tissue-contact rails, thereby storing spring energy in the first and second lateral connecting strut arrays.

2. The intravascular compliance-enhancing spring implant device of claim 1, wherein the first and second tissue-contact rails and the first and second lateral connecting strut arrays are formed of a common lattice structure comprising interconnecting struts defining open cells.

3. The intravascular compliance-enhancing spring implant device of claim 2, wherein the first and second lateral connecting strut arrays have a radially-undulating form.

4. The intravascular compliance-enhancing spring implant device of claim 1, wherein the first and second lateral connecting strut arrays each comprise a plurality of longitudinally offset, non-interconnecting spring struts with central apexed flexures.

5. The intravascular compliance-enhancing spring implant device of claim 4, wherein:
the central apexed flexures point in a first axial direction; and
the plurality of spring struts each include first and second end flexures on opposite sides of the central apexed flexures, the first and second end flexures pointing in a second axial direction opposite the first axial direction.

6. An intravascular compliance-enhancing spring implant device comprising:
first and second tissue-contact end portions configured to press against an inner diameter of a target blood vessel; and
one or more spring elements configured to be compressed in a manner as to reduce a distance between the first and second tissue-contact end portions.

7. The intravascular compliance-enhancing spring implant device of claim 6, wherein:
each of the one or more spring elements comprises a V-shaped spring;
the V-shaped spring forms a central apex connected to the first and second tissue-contact end portions by respective struts; and
the struts have first and second curved portions separated by an inflection portion.

8. The intravascular compliance-enhancing spring implant device of claim 7, wherein the struts:
project from one of the first or second tissue-contact end portions;
deflect in a first longitudinal direction on a first side of the inflection portion; and
project in a second longitudinal direction opposite the first longitudinal direction on a second side of the inflection portion towards an apex spring feature that points in the second longitudinal direction.

9. The intravascular compliance-enhancing spring implant device of claim 6, wherein:
the one or more spring elements comprises a plurality of serially-arranged spring elements;
apices of the plurality of spring elements are aligned along a lengthwise dimension of the spring implant device; and
the plurality of spring elements are each coupled at opposite ends thereof to the first and second tissue-contact end portions, respectively.

10. The intravascular compliance-enhancing spring implant device of claim 9, wherein:
the plurality of serially-arranged spring elements comprises first and second rows of spring elements; and
each spring element of the first row of spring elements is longitudinally-aligned with a corresponding spring element of the second row of spring elements.

11. The intravascular compliance-enhancing spring implant device of claim 9, wherein:
the one or more spring elements are configured to compress primarily in a first transverse dimension with respect to an axis of the spring implant device; and
the first and second tissue-contact end portions comprise first and second end spring elements, respectively, configured to compress primarily in a second transverse dimension that is orthogonal to the first transverse dimension.

12. The intravascular compliance-enhancing spring implant device of claim 9, wherein the spring implant device has an oval axial cross-sectional shape.

13. The intravascular compliance-enhancing spring implant device of claim 9, wherein:
adjacent pairs of the plurality of spring elements are longitudinally overlapping;
each of the plurality of spring elements comprises an apex; and
a longitudinally-adjacent pair of the plurality of spring elements comprises a first spring element positioned such that an apex of the first spring element is disposed in a space formed by an interior angle of an apex of a second spring element of the longitudinally-adjacent pair of the plurality of spring elements.

14. The intravascular compliance-enhancing spring implant device of claim 6, further comprising a stent frame, wherein:
the stent frame forms the first and second tissue-contact end portions and the one or more spring elements; and
the one or more spring elements comprise first and second undulating sidewalls of the stent frame.

15. The intravascular compliance-enhancing spring implant device of claim 14, wherein the first and second sidewalls are parallel in a relaxed state of the implant device.

16. The intravascular compliance-enhancing spring implant device of claim 14, wherein undulations of the first and second sidewalls comprise convex and concave bends that are configured to store spring energy when the first and second sidewalls are compressed with respect to a major-axis dimension of the stent frame.

17. The intravascular compliance-enhancing spring implant device of claim 14, wherein the first and second tissue-contact end portions comprise circumferentially-open tubes providing an outwardly-convex tissue-contact surface.

18. The intravascular compliance-enhancing spring implant device of claim 14, wherein the first and second undulating sidewalls include at least three apices.

19. An intravascular implant device for improving compliance of a native blood vessel, comprising:
a tubular scaffold having a substantially C-shaped cross-section, the tubular scaffold defining a longitudinal axis and having a major axis diameter and a minor axis diameter that is less than the major axis diameter;
first and second major-axis elbow apices at major-axis ends of the tubular scaffold;
first and second longitudinal contact edges defining a longitudinal split in the tubular scaffold, the first and second longitudinal contact edges being radially curved to present convex contact rails; and
first and second relatively flat arm wall portions extending from the first and second major-axis elbow apices and terminating at the first and second longitudinal contact edges, respectively;
wherein the tubular scaffold is configured to flex at the first and second major-axis elbow apices to store energy and return the energy to circulation in a target blood vessel where the spring implant device is implanted to increase compliance of the target blood vessel.

20. The intravascular compliance-enhancing spring implant device of claim 19, wherein the first and second longitudinal contact edges are curved radially inwardly to terminate within a lumen defined by the tubular scaffold.
